(19) United States
(12) Patent Application Publication
(10) Pub. No.: US 2008/0064714 A1
(43) Pub. Date: Mar. 13, 2008

(54) Novel Pyrrolodihydroisoquinolines

(75) Inventors: Matthias Vennemann, Konstanz (DE); Thomas Bar, Reichenau (DE); Juergen Braunger, Konstanz (DE); Volker Gekeler, Konstanz (DE); Petra Gimmnich, Konstanz (DE); Paola Ciapetti, Altdorf (FR); Jean-Marie Contreras, Benfeld (FR); Camille Georges Wermuth, Strasbourg (FR)

Correspondence Address: NATH & ASSOCIATES PLLC, 112 South West Street, Alexandria, VA, 22314, US

(73) Assignee: NYCOMED GMBH, Konstanz (DE)

(21) Appl. No.: 11/794,494

(22) PCT Filed: Jan. 11, 2006
(86) PCT No.: PCT/EP06/50165

§ 371 Date: Aug. 16, 2007

(30) Foreign Application Priority Data
Jan. 12, 2005 (EP) 05100155.0

(51) Int. Cl. A61K 31/47; A61P 35/00; C07D 217/00

(52) U.S. Cl. 514/294; 546/94

(57) ABSTRACT

The invention relates to novel pyrrolodihydroisoquinoline derivatives which are efficacious inhibitors of cellular (hyper)proliferation and/or inducers of apoptosis in cancer cells.

### FIELD OF APPLICATION OF THE INVENTION

The invention relates to novel pyrrolodihydroisoquinoline derivatives, which can be used in the pharmaceutical industry for the production of pharmaceutical compositions.

### KNOWN TECHNICAL BACKGROUND

Cancer chemotherapy was established with the alkylating agent Cyclophosphamide (Endoxan®), an oxazaphosphorin pro-drug activated preferentially in the tumor. The target of alkylating agents like Cyclophosphamide is DNA and the concept, that cancer cells with uncontrolled proliferation and a high mitotic index are killed preferentially, proved to be very sucessfull. Standard cancer chemotherapeutic drugs finally kill cancer cells upon induction of programmed cell death (“apoptosis”) by targeting basic cellular processes and molecules. These basic cellular processes and molecules include RNA/DNA (alkylating and carbamylating agents, plain analogs and topoisomerase inhibitors), metabolism (drugs of this class are named anti-metabolites and examples are folic acid, purin and pyrimidine antagonists) as well as the mitotic spindle apparatus with αβ-tubulin heterodimers as the essential component (drugs are categorized into stabilizing and destabilizing tubulin inhibitors; examples are Taxol/Paclitaxel®, Docetaxel/Taxotere® and vinca alkaloids).

The International applications WO 02/48144, WO 03/014115, WO 03/014116, WO 03/014117 and WO 03/051877 disclose pyrrolodihydroisoquinoline derivatives with PDE10 inhibitory activity. The U.S. Pat. No. 5,965,575 discloses pyrrolodihydroisoquinoline derivatives as 5HT_{1B} antagonists. The International application WO 2005/003130 relates to pyrrolodihydroisoquinoline derivatives which are efficacious inhibitors of cellular (hyper)proliferation and/or inducers of apoptosis in cancer cells. The International application WO 98/55118 describes the use of nitrogen heterocyclic aromatic derivatives in the topical treatment of the diseases of the epithelial tissues.

### DESCRIPTION OF THE INVENTION

It has now been found that the pyrroloisoquinoline derivatives, which are described in greater details below, differ from prior art compounds by unanticipated structural features and have surprising and particularly advantageous properties.

In more detail, thus, for example, it has been unexpectedly and unanticipatedly found that the pyrrolodihydroisoquinoline derivatives, which are described in greater details below, are potent and highly efficacious inhibitors of cellular proliferation and inducers of apoptosis in cancer cells. Therefore, yet unanticipatedly, these pyrrolodihydroisoquinoline derivatives can be useful for treating (hyper)proliferative diseases and/or disorders responsive to the induction of apoptosis, in particular cancer.

In this context, in further more surprising detail, it has been particularly found that the pyrrolodihydroisoquinoline derivatives, which are described in greater details below, stand out from the general class of the pyrrolodihydroisoquinolines, whose original property is inhibition of PDE10, in interesting and valuable properties, such as e.g. those mentioned afore, i.e. inhibiting cellular (hyper)proliferation and inducing apoptosis in cancer cells, which make them particularly interesting for treating e.g. (hyper)proliferative diseases and/or disorders responsive to the induction of apoptosis, in particular cancer.

The invention thus relates to compounds of formula I

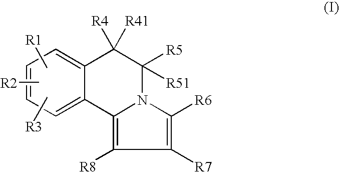

in which
R1 is halogen, nitro, amino, mono- or di-1-4C-alkylamino, 1-4C-alkyl, hydroxyl, 1-4C-alkoxy, 1-4C-alkoxy-2-4C-alkoxy, 3-7C-cycloalkoxy, 3-7C-cycloalkylmethoxy, or completely or predominantly fluorine-substituted 1-4C-alkoxy,
R2 is hydrogen, halogen or 1-4C-alkoxy,
R3 is hydrogen or 1-4C-alkoxy, or
R2 and R3 bound to the benzo ring moiety in ortho-position to each other together form a 1-2C-alkylenedioxy bridge, or
R2 and R3 bound to the benzo ring moiety in ortho-position to each other together form a completely or predominantly fluorine-substituted 1-2C-alkylenedioxy bridge, or
R1 and R2 bound to the benzo ring moiety in ortho-position to each other together form a 1-2C-alkylenedioxy bridge and R3 is hydrogen, or
R1 and R2 bound to the benzo ring moiety in ortho-position to each other together form a completely or predominantly fluorine-substituted 1-2C-alkylenedioxy bridge and R3 is hydrogen,
R4 is hydrogen, or 1-4C-alkyl,
R41 is hydrogen, or 1-4C-alkyl,
R5 is hydrogen,
R51 is hydrogen,
R6 is 1-6C-alkyl, or 1-4C-alkyl substituted by R61, in which
R61 is 1-4C-alkoxycarbonyl, or carboxyl,
R7 is phenyl, naphthyl, Har, R71- and/or R72- and/or R73-substituted phenyl, or R74-substituted Har, in which
Har is bonded to the pyrroloisoquinoline scaffold via a ring carbon atom, and is a monocyclic or fused bicyclic 5- to 10-membered partially or fully aromatic heterocyclic ring radical comprising one to four heteroatoms, each of which is selected from a group consisting of nitrogen, oxygen and sulfur,
R71 is hydroxyl, halogen, nitro, cyano, trifluoromethyl, 1-4C-alkyl, 1-4C-alkoxy, 3-7C-cycloalkoxy, 3-7C-cycloalkylmethoxy, amino, mono- or di-1-4C-alkylamino, 1-4C-alkylsulphonylamino, arylsulphonylamino, mono- or di-1-4C-alkylaminocarbonyl, completely or predominantly fluorine-substituted 1-4C-alkoxy, carbamoyl, tetrazolyl, 1-4C-alkoxycarbonyl, carboxyl, aryl, aryloxy, or —N(H)S(O)₂—N(R712)R713, in which
aryl is phenyl or R711-substituted phenyl, in which
R711 is halogen, 1-4C-alkyl, 1-4C-alkoxy, nitro or cyano,
R712 is 1-4C-alkyl,
R713 is 1-4C-alkyl, or
R712 and R713 together and with inclusion of the nitrogen atom to which they are bound form a radical Het, in which
Het is pyrrolidin-1-yl, piperidin-1-yl or morpholin-4-yl,
R72 is halogen, 1-4C-alkyl, or 1-4C-alkoxy,
R73 is 1-4C-alkyl, or 1-4C-alkoxy,
R74 is halogen, 1-4C-alkyl, trifluoromethyl, 1-4C-alkoxy, cyano, amino, mono- or di-1-4C-alkylamino, 1-4C-alkoxycarbonyl, morpholino, carboxyl, nitro, phenyl, phenoxy, phenyl-1-4C-alkyl, arylsulphonyl, 1-4C-alkylsulphonyl, or —S(O)₂—N(R712)R713,
R8 is —C(O)—R9, in which
R9 is 1-4C-alkyl, 3-7C-cycloalkyl, 3-7C-cycloalkyl-1-4C-alkyl, or phenyl-1-4C-alkyl,

and the stereoisomers as well as the salts of these compounds and stereoisomers.

1-4C-Alkyl represents a straight-chain or branched alkyl radical having 1 to 4 carbon atoms. Examples which may be mentioned are the butyl, isobutyl, sec-butyl, tert-butyl, propyl, isopropyl and preferably the ethyl and methyl radicals.

1-6C-Alkyl represents a straight-chain or branched alkyl radical having 1 to 6 carbon atoms. Examples which may be mentioned are the hexyl, isohexyl(4-methylpentyl), neohexyl(3,3-dimethylbutyl), pentyl, isopentyl(3-methylbutyl), neopentyl(2,2-dimethylpropyl), butyl, isobutyl, sec-butyl, tert-butyl, propyl, isopropyl, ethyl or methyl radicals.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

2-4C-Alkoxy represents radicals which, in addition to the oxygen atom, contain a straight-chain or branched alkyl radical having 2 to 4 carbon atoms. Examples which may be mentioned are the butoxy, isobutoxy, sec-butoxy, tert-butoxy, propoxy, isopropoxy and preferably the ethoxy radical.

3-7C-Cycloalkoxy represents cyclopropyloxy, cyclobutyloxy, cyclopentyloxy, cyclohexyloxy and cycloheptyloxy, of which cyclopropyloxy and cyclopentyloxy are to be emphasized.

3-7C-Cycloalkyl represents cyclopropyl, cyclobutyl, cyclopentyl, cyclohexyl and cycloheptyl, of which cyclopropyl and cyclopentyl are to be emphasized.

3-7C-Cycloalkylmethoxy represents cyclopropylmethoxy, cyclobutylmethoxy, cyclopentylmethoxy, cyclohexylmethoxy and cycloheptylmethoxy, of which cyclopropylmethoxy and cyclopentylmethoxy are to be emphasized.

3-7C-Cycloalkyl-1-4C-alkyl represents one of the abovementioned 1-4C-alkyl radicals, which is substituted by one of the abovementioned 3-7C-cycloalkyl radicals. Examples which may be mentioned are the cyclopropylmethyl, the cyclohexylethyl and the cyclohexylmethyl radicals.

As completely or predominantly fluorine-substituted 1-4C-alkoxy, for example, the 2,2,3,3,3-penta-fluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals may be mentioned. “Predominantly” in this connection means that more than half of the hydrogen atoms of the 1-4C-alkoxy radicals are replaced by fluorine atoms.

1-4C-Alkoxy-2-4C-alkoxy represents one of the abovementioned 2-4C-alkoxy radicals, which is substituted by one of the abovementioned 1-4C-alkoxy radicals. Examples which may be mentioned are the 2-methoxyethoxy, 2-ethoxyethoxy and the 2-isopropoxyethoxy radicals.

1-2C-Alkylenedioxy represents, for example, the methylenedioxy [—O—CH₂—O—] and the ethylenedioxy [—O—CH₂—CH₂—O—] radicals.

As completely or predominantly fluorine-substituted 1-2C-alkylenedioxy bridge, for example, the difluoromethylenedioxy [—O—CF₂—O—] radical may be mentioned. “Predominantly” in this connection means that more than half of the hydrogen atoms of the 1-4C-alkylenedioxy radical are replaced by fluorine atoms.

Phenyl-1-4C-alkyl stands for one of the abovementioned 1-4C-alkyl radicals, which is substituted by a phenyl radical. Examples which may be mentioned are the phenethyl and the benzyl radicals.

1-4C-Alkoxycarbonyl represents a radical which, in addition to the carbonyl group, contains one of the abovementioned 1-4C-alkoxy radicals. Examples which may be mentioned are the methoxycarbonyl and ethoxycarbonyl radicals.

Halogen within the meaning of the invention is iodine and, particularly, bromine, chlorine and fluorine.

In addition to the nitrogen atom, mono- or di-1-4C-alkylamino radicals contain one or two of the abovementoned 1-4C-alkyl radicals. Di-1-4C-alkylamino is to be emphasized and here, in particular, dimethyl-, diethyl- and diisopropylamino.

1-4C-Alkylsulfonyl is a sulfonyl group to which one of the abovementioned 1-4C-alkyl radicals is bonded. An example is the methanesulfonyl radical (CH₃SO₂—).

1-4C-Alkylsulfonylamino is an amino group which is substituted by one of the abovementioned 1-4C-alkylsulfonyl radicals. An example is the methanesulfonylamino radical (CH₃SO₂NH—).

Aryl radicals referred to herein, including those forming part of other groups or radicals, include phenyl or R711-substituted phenyl radicals.

Aryloxy stands for phenoxy or R711-substituted phenoxy.

Mono- or Di-1-4C-alkylaminocarbonyl radicals contain in addition to the carbonyl group one of the abovementioned mono- or di-1-4C-alkylamino radicals. Examples which may be mentioned are the N-methyl- the N,N-dimethyl-, the N-ethyl-, the N-propyl-, the N,N-diethyl- and the N-isopropylaminocarbonyl radical.

Har refers to a monocyclic or fused bicyclic 5- to 10-membered partially or fully aromatic heterocyclic ring or ring system comprising one to four, particularly one to three, heteroatoms, each of which is selected from a group consisting of nitrogen, oxygen and sulphur.

The Har radical is bonded via a ring carbon atom to the adjacent pyrroloisoquinoline scaffold.

In one embodiment (embodiment a) Har refers to a monocyclic 5-membered fully aromatic heteroaryl radical comprising one to four heteroatoms, each of which is selected from a group consisting of nitrogen, oxygen and sulphur,

Exemplary Har radicals according to embodiment a may include, without being restricted to, furanyl, thiophenyl, pyrrolyl, oxazolyl, isoxazolyl, thiazolyl, isothiazolyl, imidazolyl, pyrazolyl, triazolyl, thiadiazolyl or oxadiazolyl.

In another embodiment (embodiment b) Har refers to a monocyclic 6-membered fully aromatic heteroaryl radical comprising one or two nitrogen atoms.

Exemplary Har radicals according to embodiment b may include pyridinyl, pyrimidinyl, pyrazinyl or pyridazinyl.

A Har radical according to embodiment a worthy to be mentioned is pyridinyl, such as e.g. pyridin-4-yl.

In another embodiment (embodiment c) Har refers to a fused bicyclic 9- or 10-membered fully aromatic heteroaryl radical comprising one to four, in particular one to three, in more particular one or two, heteroatoms, each of which is selected from a group consisting of nitrogen, oxygen and sulphur.

Exemplary Har radicals according to embodiment c may include, without being restricted to, the benzo-fused analogues of the Har radicals mentioned exemplarily above in embodiment a or b, such as, for example, quinazolinyl, quinoxalinyl, cinnolinyl, quinolyl, isoquinolyl, indolyl, isoindolyl, indazolyl, benzothiophenyl, benzofuranyl, benzoxazolyl, benzothiazolyl or benzimidazolyl; or naphthyridinyl, phthalazinyl, imidazopyridinyl, purinyl, pteridinyl or imidazopyridazinyl.

The Har radicals according to embodiment c, which contain a benzene ring, can be attached to the parent molecular group via any ring carbon atom of the heteroatom containing ring or of the benzene ring.

Har radicals according to embodiment b worthy to be mentioned are indolyl, benzothiophenyl, or quinolinyl, such as e.g. indol-3-yl, benzothiophen-3-yl, or quinolin-4-yl.

In another embodiment (embodiment d) Har refers to a bicyclic partially aromatic heterocyclic radical made up of

a first constituent being a 5- or 6-membered monocyclic fully saturated heterocyclic ring,
which heterocyclic ring comprises one or two heteroatoms independently selected from nitrogen, oxygen and sulphur,

and, fused to said first constituent,

a second constituent being benzene ring,

whereby said Har ring system is attached to the parent molecular group via any ring carbon atom of the benzene moiety.

Exemplary Har radicals according to embodiment d may include, without being restricted to, indolinyl, isoindolinyl, 1,2,3,4-tetrahydroquinolinyl, 1,2,3,4-tetrahydroisoquinolinyl, 1,3-benzodioxolyl, 2,3-dihydro-1,4-benzodioxinyl, 2,3-dihydrobenzothiophenyl, 2,3-dihydrobenzofuranyl, or chromanyl.

In another embodiment (embodiment e) Har refers to a stabile N-oxide derivative of any nitrogen-containing heteroaryl ring, particularly of any imino type nitrogen (═N—) containing heteroaryl ring, according to embodiment a or b.

Exemplary Har radicals according to embodiment d may include, without being restricted to, N-oxy-pyridinyl.

A Har radical according to embodiment c in particular worthy to be mentioned is 1N-oxy-pyridin-4-yl.

Naphthyl includes naphthalen-1-yl and naphthalen-2-yl.

The term Har includes all the possible isomeric forms thereof, in particular the positional isomers thereof. Such as, for example, pyridinyl or pyridyl includes pyridin-2-yl, pyridin-3-yl and pyridin-4-yl.

Constituents which are substituted as described herein may be substituted, unless otherwise noted, at any possible position.

The substituents R1, R2 and/or R3 may be attached, unless otherwise noted, at any position of the benzo moiety of the pyrrolodihydroisoquinoline ring.

Har may be substituted by its substituents as mentioned herein at any possible position, such as e.g. at any substitutable ring carbon or ring nitrogen atom.

Har rings containing imino-type ring nitrogen atoms (—N═) may be preferably not substituted (i.e. quaternized) on these imino-type ring nitrogen atoms by the mentioned substituents.

When any variable occurs more than one time in any constituent, each definition is independent.

Suitable salts for compounds of the formula I—depending on substitution—are all acid addition salts or all salts with bases. Particular mention may be made of the pharmacologically tolerable inorganic and organic acids and bases customarily used in pharmacy. Those suitable are, on the one hand, water-insoluble and, particularly, water-soluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in salt preparation—depending on whether a mono- or polybasic acid is concerned and depending on which salt is desired—in an equimolar quantitative ratio or one differing therefrom.

On the other hand, salts with bases are—depending on substitution—also suitable. As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

Pharmacologically intolerable salts, which can be obtained, for example, as process products during the preparation of the compounds of formula I according to the invention on an industrial scale, are converted into pharmacologically tolerable salts by processes known to the person skilled in the art.

According to expert's knowledge the compounds of formula I of the invention as well as their salts may contain, e.g. when isolated in crystalline form, varying amounts of solvents. Included within the scope of the invention are therefore all solvates and in particular all hydrates of the compounds of formula I as well as all solvates and in particular all hydrates of the salts of the compounds of formula I.

Depending on substitution the compounds of formula I can be chiral compounds having, for example, chiral centers and/or chiral axes due to hindered rotation about single bonds. Chiral axes can be present in particular in those compounds according to the invention, in which R7 is a bicyclic ring, or a monocyclic ring substituted in the ortho position with respect to the binding position in which said monocyclic ring is bonded to the pyrrolo[2.1-a]isoquinoline ring system. A chiral center can be, for example, -depending on the meaning of R4 and R41-located at position 6 of the pyrrolo[2.1-a]isoquinolin scaffold. The invention therefore includes all conceivable pure diastereomers and pure enantiomers and mixtures thereof in any mixing ratio including the racemates, as well as the salts thereof. The diastereomer mixtures can be separated into the individual isomers by standard methods, e.g. by chromatographic processes. The enantiomers can be separated in a known manner (e.g. by chromatographic processes on chiral phases or by resolution).

Therefore, e.g. the pure (6R)- and the pure (6S)-enantiomers, as well as mixtures thereof in any mixing ratio including the racemates, and the salts thereof, are part of this invention.

In the context of this invention, hyperproliferation and analogous terms are used to describe aberrant/dysregulated cellular growth, a hallmark of diseases like cancer. This hyperproliferation might be caused by single or multiple cellular/molecular alterations in respective cells and can be, in context of a whole organism, of benign or malignant behaviour. Inhibition of cell proliferation and analogous terms is used to denote an ability of the compound to retard the growth of and/or kill a cell contacted with that compound as compared to cells not contacted with that compound. Most preferable this inhibition of cell proliferation is 100%, meaning that proliferation of all cells is stopped and/or cells undergo programmed cell death/apoptosis. In some preferred embodiments the contacted cell is a neoplastic cell. A neoplastic cell is defined as a cell with aberrant cell proliferation. A benign neoplasia is described by hyperproliferation of cells, incapable of forming an aggressive, metastasizing tumor in-vivo. In contrast, a malignant neoplasia is described by cells with different cellular and biochemical abnormalities, e.g. capable of forming tumor metastasis. The acquired functional abnormalities of malignant neoplastic cells (also defined as “hallmarks of cancer”) are replicative potential (“hyperproliferation”), self-sufficiency in growth signals, insensitivity to anti-growth signals, evasion from apoptosis, sustained angiogenesis and tissue invasion and metastasis.

Inducer of apoptosis and analogous terms are used to identify a compound which excecutes programmed cell death in cells contacted with that compound. Apoptosis is defined by complex biochemical events within the contacted cell, such as the activation of cystein specific proteinases (“caspases”) and the fragmentation of chromatin. Induction of apoptosis in cells contacted with the compound might not necessarily coupled with inhibition of cell proliferation. Preferably, the inhibition of cell proliferation and/or induction of apoptosis is specific to cells with aberrant cell growth (hyperproliferation). Thus, compared to cells with aberrant cell growth, normal proliferating or arrested cells are less sensitive or even insensitive to the proliferation inhibiting or apoptosis inducing activity of the compound. Finally, cytotoxic is used in a more general sense to identify compounds which kill cells by various mechanisms, including the induction of apoptosis/programmed cell death in a cell cycle dependent or cell-cycle independent manner.

Compounds according to this invention more worthy to be mentioned are those compounds of formula I, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is hydrogen, halogen or 1-4C-alkoxy,
R3 is hydrogen or 1-4C-alkoxy,
R4 is hydrogen, or 1-4C-alkyl,
R41 is hydrogen, or 1-4C-alkyl,
R5 is hydrogen,
R51 is hydrogen,
R6 is 1-6C-alkyl, or 1-4C-alkyl substituted by R61, in which
R61 is 1-4C-alkoxycarbonyl, or carboxyl,
R7 is phenyl, naphthyl, Har, R71- and/or R72- and/or R73-substituted phenyl, or R74-substituted Har, in which
Har is bonded to the pyrroloisoquinoline scaffold via a ring carbon atom, and is a monocyclic or fused bicyclic 5- to 10-membered partially or fully aromatic heterocyclic ring radical comprising one to four heteroatoms, each of which is selected from a group consisting of nitrogen, oxygen and sulfur,
R71 is hydroxyl, halogen, nitro, 1-4C-alkoxy, amino, mono- or di-1-4C-alkylamino, carbamoyl, or aryl, in which
aryl is phenyl, or R711-substituted phenyl, in which
R711 is halogen, 1-4C-alkyl, 1-4C-alkoxy, nitro or cyano,
R72 is 1-4C-alkyl, or 1-4C-alkoxy,
R73 is 1-4C-alkyl, or 1-4C-alkoxy,
R74 is 1-4C-alkyl,
R8 is —C(O)—R9, in which
R9 is 1-4C-alkyl, 3-7C-cycloalkyl, 3-7C-cycloalkyl-1-4C-alkyl, or phenyl-1-4C-alkyl,

and the stereoisomers as well as the salts of these compounds and stereoisomers.

Compounds according to this invention in particular worthy to be mentioned are those compounds of formula I, in which

either
R1 is nitro, amino, 1-4C-alkyl, 1-4C-alkoxy, 1-4C-alkoxy-2-4C-alkoxy, or completely or predominantly fluorine-substituted 1-4C-alkoxy, and
R2 is 1-4C-alkoxy,
or
R1 is 1-4C-alkoxy, or 1-4C-alkoxy-2-4C-alkoxy, and
R2 is halogen,
R3 is hydrogen,
whereby none of R1, R2 and R3 is bound to the 10-position of the pyrrolo[2.1-a]isoquinoline ring,
R4 is hydrogen, or 1-4C-alkyl,
R41 is hydrogen, or 1-4C-alkyl,
R5 is hydrogen,
R51 is hydrogen,
R6 is 1-6C-alkyl, or 1-4C-alkyl substituted by R61, in which
R61 is 1-4C-alkoxycarbonyl, or carboxyl,
R7 is naphthyl, Har, R71- and/or R72- and/or R73-substituted phenyl, or R74-substituted Har, in which
Har is either
   a monocyclic 5-membered heteroaryl radical comprising one to four heteroatoms, each of which is selected from a group consisting of nitrogen, oxygen and sulfur,
   or
   a monocyclic 6-membered heteroaryl radical comprising one or two nitrogen atoms,
   or
   a fused bicyclic 9- or 10-membered heteroaryl comprising one to three heteroatoms, each of which is selected from a group consisting of nitrogen, oxygen and sulfur,
   or
   N-oxy-pyridyl,
R71 is hydroxyl, halogen, nitro, 1-4C-alkoxy, amino, mono- or di-1-4C-alkylamino, carbamoyl, or aryl, in which
aryl is phenyl, or R711-substituted phenyl, in which
R711 is halogen, 1-4C-alkyl, 1-4C-alkoxy, nitro or cyano,
R72 is 1-4C-alkyl, or 1-4C-alkoxy,
R73 is 1-4C-alkyl, or 1-4C-alkoxy,
R74 is 1-4C-alkyl,
R8 is —C(O)—R9, in which
R9 is 1-4C-alkyl, or 3-7C-cycloalkyl,

and the stereoisomers as well as the salts of these compounds and stereoisomers.

Compounds according to this invention in more particular worthy to be mentioned are those compounds of formula I, in which

either
R1 is 1-4C-alkoxy, 1-4C-alkoxy-2-4C-alkoxy, or completely or predominantly fluorine-substituted 1-4C-alkoxy, and
R2 is 1-4C-alkoxy,
or
R1 is 1-4C-alkoxy, or 1-4C-alkoxy-2-4C-alkoxy, and
R2 is fluorine or chlorine,
R3 is hydrogen,
whereby none of R1, R2 and R3 is bound to the 10-positon of the pyrrolo[2.1-a]isoquinoline ring,
R4 is hydrogen, or 1-4C-alkyl,
R41 is hydrogen, or 1-4C-alkyl,
R5 is hydrogen,
R51 is hydrogen,
R6 is 1-4C-alkyl, or 1-4C-alkyl substituted by R61, in which
R61 is 1-4C-alkoxycarbonyl, or carboxyl,
R7 is naphthyl, Har, or R71- and/or R72- and/or R73-substituted phenyl, in which
Har is a fused bicyclic 9- or 10-membered heteroaryl radical comprising one to three heteroatoms, each of which is selected from a group consisting of nitrogen, oxygen and sulfur,
R71 is hydroxyl, halogen, di-1-4C-alkylamino, or aryl, in which
aryl is phenyl, or R711-substituted phenyl, in which
R711 is halogen, 1-4C-alkyl, 1-4C-alkoxy, nitro or cyano,
R72 is 1-4C-alkyl, or 1-4C-alkoxy,
R73 is 1-4C-alkyl, or 1-4C-alkoxy,
R8 is —C(O)—R9, in which
R9 is 1-4C-alkyl, or 3-5C-cycloalkyl,

and the stereoisomers as well as the salts of these compounds and stereoisomers.

Compounds according to this invention to be emphasized are those compounds of formula I, in which either
R1 is 1-2C-alkoxy, 1-2C-alkoxy-2-3C-alkoxy, or completely or predominantly fluorine-substituted 1-2C-alkoxy, and
R2 is 1-2C-alkoxy,
or
R1 is 1-2C-alkoxy, and
R2 is fluorine or chlorine,
R3 is hydrogen,

whereby none of R1, R2 and R3 is bound to the 10-positon of the pyrrolo[2.1-a]isoquinoline ring,
R4 is hydrogen, or methyl,
R41 is hydrogen, or methyl,
R5 is hydrogen,
R51 is hydrogen,
R6 is methyl,
R7 is either
   naphthyl, such as e.g. napthalen-1-yl,
   or
   dimethyamino-phenyl, such as e.g. 3-dimethyamino-phenyl,
   or
   4-hydroxy-3,5-dimethylphenyl,
   or
   2-fluoro-3,4-dimethoxy-phenyl,
   or
   3,4,5-trimethoxy-phenyl,
   or
   1,1′-biphen-4-yl,
   or
   Har, in which
Har is a fused bicyclic 9- or 10-membered heteroaryl comprising a benzene ring and one or two heteroatoms, each of which is selected from a group consisting of nitrogen, oxygen and sulfur, such as, for example, indolyl, benzothiophenyl or quinolinyl, e.g. indol-3-yl, benzothiophen-3-yl or quinolin4-yl,
R8 is —C(O)—R9, in which
R9 is 1-4C-alkyl especially 1-2C-alkyl, or cyclopropyl,

and the stereoisomers as well as the salts of these compounds and stereoisomers.

As exemplary compounds according to this invention the following compounds of formula Ia

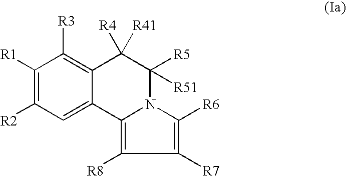

in which
R5 and R51 are both hydrogen, and
R6 is methyl,
R7 is 3-dimethyamino-phenyl,

and the stereoisomers as well as the salts of these compounds and stereoisomers,

may be mentioned by means of the substituent meanings for R1, R2, R3, R4, R41 and R8 in the Table 1 given below.

As further exemplary compounds according to this invention the following compounds of formula Ia in which
R5 and R51 are both hydrogen,
R6 is methyl, and
R7 is quinolin-4-yl,

and the stereoisomers as well as the salts of these compounds and stereoisomers,

may be mentioned by means of the substituent meanings for R1, R2, R3, R4, R41 and R8 in the Table 1 given below.

As further exemplary compounds according to this invention the following compounds of formula Ia in which
R5 and R51 are both hydrogen,
R6 is methyl, and
R7 is indol-3-yl,

and the stereoisomers as well as the salts of these compounds and stereoisomers,

may be mentioned by means of the substituent meanings for R1, R2, R3, R4, R41 and R8 in the Table 1 given below.

As further exemplary compounds according to this invention the following compounds of formula Ia in which
R5 and R51 are both hydrogen,
R6 is methyl, and
R7 is benzofuran-3-yl,

and the stereoisomers as well as the salts of these compounds and stereoisomers,

may be mentioned by means of the substituent meanings for R1, R2, R3, R4, R41 and R8 in the Table 1 given below.

As further exemplary compounds according to this invention the following compounds of formula Ia in which
R5 and R51 are both hydrogen,
R6 is methyl, and
R7 is benzothiophen-3-yl,

and the stereoisomers as well as the salts of these compounds and stereoisomers,

may be mentioned by means of the substituent meanings for R1, R2, R3, R4, R41 and R8 in the Table 1 given below.

As further exemplary compounds according to this invention the following compounds of formula Ib

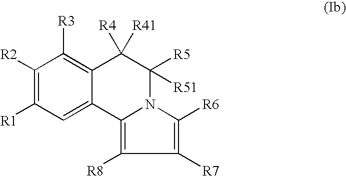

in which
R5 and R51 are both hydrogen, and
R6 is methyl,
R7 is 3-dimethyamino-phenyl,

and the stereoisomers as well as the salts of these compounds and stereoisomers,

may be mentioned by means of the substituent meanings for R1, R2, R3, R4, R41, R7 and R8 in the Table 1 given below.

As further exemplary compounds according to this invention the following compounds of formula Ib in which
R5 and R51 are both hydrogen,
R6 is methyl, and
R7 is quinolin-4-yl,

and the stereoisomers as well as the salts of these compounds and stereoisomers,

may be mentioned by means of the substituent meanings for R1, R2, R3, R4, R41 and R8 in the Table 1 given below.

As further exemplary compounds according to this invention the following compounds of formula Ib in which
R5 and R51 are both hydrogen,
R6 is methyl, and
R7 is indol-3-yl,

and the stereoisomers as well as the salts of these compounds and stereoisomers,

may be mentioned by means of the substituent meanings for R1, R2, R3, R4, R41 and R8 in the Table 1 given below.

As further exemplary compounds according to this invention the following compounds of formula Ib in which
R5 and R51 are both hydrogen,
R6 is methyl, and
R7 is benzofuran-3-yl,

and the stereoisomers as well as the salts of these compounds and stereoisomers,

may be mentioned by means of the substituent meanings for R1, R2, R3, R4, R41 and R8 in the Table 1 given below.

As further exemplary compounds according to this invention the following compounds of formula Ib in which
R5 and R51 are both hydrogen,
R6 is methyl, and
R7 is benzothiophen-3-yl,

and the stereoisomers as well as the salts of these compounds and stereoisomers,

may be mentioned by means of the substituent meanings for R1, R2, R3, R4, R41 and R8 in the Table 1 given below.

As other exemplary compounds according to this invention the following compounds of formula Ia in which
R5 and R51 are both hydrogen, and
R6 is methyl,
R7 is 3-dimethyamino-phenyl,

and the stereoisomers as well as the salts of these compounds and stereoisomers,

may be mentioned by means of the substituent meanings for R1, R2, R3, R4, R41 and R8 in the Table 2 given below.

As other exemplary compounds according to this invention the following compounds of formula Ia in which
R5 and R51 are both hydrogen,
R6 is methyl, and
R7 is quinolin-4-yl,

and the stereoisomers as well as the salts of these compounds and stereoisomers,

may be mentioned by means of the substituent meanings for R1, R2, R3, R4, R41 and R8 in the Table 2 given below.

As other exemplary compounds according to this invention the following compounds of formula Ia in which
R5 and R51 are both hydrogen,
R6 is methyl, and
R7 is indol-3-yl,

and the stereoisomers as well as the salts of these compounds and stereoisomers,

may be mentioned by means of the substituent meanings for R1, R2, R3, R4, R41 and R8 in the Table 2 given below.

As other exemplary compounds according to this invention the following compounds of formula Ia in which
R5 and R51 are both hydrogen,
R6 is methyl, and
R7 is benzofuran-3-yl,

and the stereoisomers as well as the salts of these compounds and stereoisomers,

may be mentioned by means of the substituent meanings for R1, R2, R3, R4, R41 and R8 in the Table 2 given below.

As other exemplary compounds according to this invention the following compounds of formula Ia in which
R5 and R51 are both hydrogen,
R6 is methyl, and
R7 is benzothiophen-3-yl,

and the stereoisomers as well as the salts of these compounds and stereoisomers,

may be mentioned by means of the substituent meanings for R1, R2, R3, R4, R41 and R8 in the Table 2 given below.

As other exemplary compounds according to this invention the following compounds of formula Ib in which
R5 and R51 are both hydrogen, and
R6 is methyl,
R7 is 3-dimethyamino-phenyl,

and the stereoisomers as well as the salts of these compounds and stereoisomers,

may be mentioned by means of the substituent meanings for R1, R2, R3, R4, R41, R7 and R8 in the Table 2 given below.

As other exemplary compounds according to this invention the following compounds of formula Ib in which
R5 and R51 are both hydrogen,
R6 is methyl, and
R7 is quinolin-4-yl,

and the stereoisomers as well as the salts of these compounds and stereoisomers,

may be mentioned by means of the substituent meanings for R1, R2, R3, R4, R41 and R8 in the Table 2 given below.

As other exemplary compounds according to this invention the following compounds of formula Ib in which
R5 and R51 are both hydrogen,
R6 is methyl, and
R7 is indol-3-yl,

and the stereoisomers as well as the salts of these compounds and stereoisomers,

may be mentioned by means of the substituent meanings for R1, R2, R3, R4, R41 and R8 in the Table 2 given below.

As other exemplary compounds according to this invention the following compounds of formula Ib in which
R5 and R51 are both hydrogen,
R6 is methyl, and
R7 is benzofuran-3-yl,

and the stereoisomers as well as the salts of these compounds and stereoisomers,

may be mentioned by means of the substituent meanings for R1, R2, R3, R4, R41 and R8 in the Table 2 given below.

As other exemplary compounds according to this invention the following compounds of formula Ib in which
R5 and R51 are both hydrogen,
R6 is methyl, and
R7 is benzothiophen-3-yl,

and the stereoisomers as well as the salts of these compounds and stereoisomers,

may be mentioned by means of the substituent meanings for R1, R2, R3, R4, R41 and R8 in the Table 2 given below.

TABLE 1

| R1 | R2 | R3 | R4 | R41 | R8 |
|---|---|---|---|---|---|
| —OCH₃ | —OCH₃ | H | H | H | ethylcarbonyl |
| —OCF₂H | —OCH₃ | H | H | H | ethylcarbonyl |
| —Cl | —OCH₃ | H | H | H | ethylcarbonyl |
| —F | —OCH₃ | H | H | H | ethylcarbonyl |
| —O(CH₂)₂OCH₃ | —OCH₃ | H | H | H | ethylcarbonyl |
| —OCH₂CH₃ | —OCH₃ | H | H | H | ethylcarbonyl |
| —OCH₃ | —OCH₃ | H | H | H | cyclopropyl- |
| | | | | | carbonyl |
| —OCF₂H | —OCH₃ | H | H | H | cyclopropyl- |
| | | | | | carbonyl |
| —Cl | —OCH₃ | H | H | H | cyclopropyl- |
| | | | | | carbonyl |
| —F | —OCH₃ | H | H | H | cyclopropyl- |
| | | | | | carbonyl |
| —O(CH₂)₂OCH₃ | —OCH₃ | H | H | H | cyclopropyl- |
| | | | | | carbonyl |
| —OCH₂CH₃ | —OCH₃ | H | H | H | cyclopropyl- |
| | | | | | carbonyl |
| —OCH₃ | —OCH₃ | H | —CH₃ | H | ethylcarbonyl |
| —OCF₂H | —OCH₃ | H | —CH₃ | H | ethylcarbonyl |
| —Cl | —OCH₃ | H | —CH₃ | H | ethylcarbonyl |
| —F | —OCH₃ | H | —CH₃ | H | ethylcarbonyl |
| —O(CH₂)₂OCH₃ | —OCH₃ | H | —CH₃ | H | ethylcarbonyl |
| —OCH₂CH₃ | —OCH₃ | H | —CH₃ | H | ethylcarbonyl |
| —OCH₃ | —OCH₃ | H | —CH₃ | H | cyclopropyl- |
| | | | | | carbonyl |
| —OCF₂H | —OCH₃ | H | —CH₃ | H | cyclopropyl- |
| | | | | | carbonyl |
| —Cl | —OCH₃ | H | —CH₃ | H | cyclopropyl- |
| | | | | | carbonyl |
| —F | —OCH₃ | H | —CH₃ | H | cyclopropyl- |
| | | | | | carbonyl |
| —O(CH₂)₂OCH₃ | —OCH₃ | H | —CH₃ | H | cyclopropyl- |
| | | | | | carbonyl |
| —OCH₂CH₃ | —OCH₃ | H | —CH₃ | H | cyclopropyl- |
| | | | | | carbonyl |
| —OCH₃ | —OCH₃ | H | —CH₃ | —CH₃ | ethylcarbonyl |
| —OCF₂H | —OCH₃ | H | —CH₃ | —CH₃ | ethylcarbonyl |
| —Cl | —OCH₃ | H | —CH₃ | —CH₃ | ethylcarbonyl |
| —F | —OCH₃ | H | —CH₃ | —CH₃ | ethylcarbonyl |
| —O(CH₂)₂OCH₃ | —OCH₃ | H | —CH₃ | —CH₃ | ethylcarbonyl |
| —OCH₂CH₃ | —OCH₃ | H | —CH₃ | —CH₃ | ethylcarbonyl |
| —OCH₃ | —OCH₃ | H | —CH₃ | —CH₃ | cyclopropyl- |
| | | | | | carbonyl |
| —OCF₂H | —OCH₃ | H | —CH₃ | —CH₃ | cyclopropyl- |
| | | | | | carbonyl |
| —Cl | —OCH₃ | H | —CH₃ | —CH₃ | cyclopropyl- |
| | | | | | carbonyl |
| —F | —OCH₃ | H | —CH₃ | —CH₃ | cyclopropyl- |
| | | | | | carbonyl |
| —O(CH₂)₂OCH₃ | —OCH₃ | H | —CH₃ | —CH₃ | cyclopropyl- |
| | | | | | carbonyl |
| —OCH₂CH₃ | —OCH₃ | H | —CH₃ | —CH₃ | cyclopropyl- |
| | | | | | carbonyl |

TABLE 2

| R1 | R2 | R3 | R4 | R41 | R8 |
|---|---|---|---|---|---|
| —OCH₃ | —OCH₂CH₃ | H | H | H | ethylcarbonyl |
| —OCF₂H | —OCH₂CH₃ | H | H | H | ethylcarbonyl |
| —Cl | —OCH₂CH₃ | H | H | H | ethylcarbonyl |
| —F | —OCH₂CH₃ | H | H | H | ethylcarbonyl |
| —O(CH₂)₂OCH₃ | —OCH₂CH₃ | H | H | H | ethylcarbonyl |
| —OCH₂CH₃ | —OCH₂CH₃ | H | H | H | ethylcarbonyl |
| —OCH₃ | —OCH₂CH₃ | H | H | H | cyclopropyl- |
| | | | | | carbonyl |
| —OCF₂H | —OCH₂CH₃ | H | H | H | cyclopropyl- |
| | | | | | carbonyl |
| —Cl | —OCH₂CH₃ | H | H | H | cyclopropyl- |
| | | | | | carbonyl |
| —F | —OCH₂CH₃ | H | H | H | cyclopropyl- |
| | | | | | carbonyl |
| —O(CH₂)₂OCH₃ | —OCH₂CH₃ | H | H | H | cyclopropyl- |
| | | | | | carbonyl |
| —OCH₂CH₃ | —OCH₂CH₃ | H | H | H | cyclopropyl- |
| | | | | | carbonyl |
| —OCH₃ | —OCH₂CH₃ | H | —CH₃ | H | ethylcarbonyl |
| —OCF₂H | —OCH₂CH₃ | H | —CH₃ | H | ethylcarbonyl |
| —Cl | —OCH₂CH₃ | H | —CH₃ | H | ethylcarbonyl |
| —F | —OCH₂CH₃ | H | —CH₃ | H | ethylcarbonyl |
| —O(CH₂)₂OCH₃ | —OCH₂CH₃ | H | —CH₃ | H | ethylcarbonyl |
| —OCH₂CH₃ | —OCH₂CH₃ | H | —CH₃ | H | ethylcarbonyl |
| —OCH₃ | —OCH₂CH₃ | H | —CH₃ | H | cyclopropyl- |
| | | | | | carbonyl |
| —OCF₂H | —OCH₂CH₃ | H | —CH₃ | H | cyclopropyl- |
| | | | | | carbonyl |
| —Cl | —OCH₂CH₃ | H | —CH₃ | H | cyclopropyl- |
| | | | | | carbonyl |
| —F | —OCH₂CH₃ | H | —CH₃ | H | cyclopropyl- |
| | | | | | carbonyl |
| —O(CH₂)₂OCH₃ | —OCH₂CH₃ | H | —CH₃ | H | cyclopropyl- |
| | | | | | carbonyl |
| —OCH₂CH₃ | —OCH₂CH₃ | H | —CH₃ | H | cyclopropyl- |
| | | | | | carbonyl |
| —OCH₃ | —OCH₂CH₃ | H | —CH₃ | —CH₃ | ethylcarbonyl |
| —OCF₂H | —OCH₂CH₃ | H | —CH₃ | —CH₃ | ethylcarbonyl |
| —Cl | —OCH₂CH₃ | H | —CH₃ | —CH₃ | ethylcarbonyl |
| —F | —OCH₂CH₃ | H | —CH₃ | —CH₃ | ethylcarbonyl |
| —O(CH₂)₂OCH₃ | —OCH₂CH₃ | H | —CH₃ | —CH₃ | ethylcarbonyl |
| —OCH₂CH₃ | —OCH₂CH₃ | H | —CH₃ | —CH₃ | ethylcarbonyl |
| —OCH₃ | —OCH₂CH₃ | H | —CH₃ | —CH₃ | cyclopropyl- |
| | | | | | carbonyl |
| —OCF₂H | —OCH₂CH₃ | H | —CH₃ | —CH₃ | cyclopropyl- |
| | | | | | carbonyl |
| —Cl | —OCH₂CH₃ | H | —CH₃ | —CH₃ | cyclopropyl- |
| | | | | | carbonyl |
| —F | —OCH₂CH₃ | H | —CH₃ | —CH₃ | cyclopropyl- |
| | | | | | carbonyl |
| —O(CH₂)₂OCH₃ | —OCH₂CH₃ | H | —CH₃ | —CH₃ | cyclopropyl- |
| | | | | | carbonyl |
| —OCH₂CH₃ | —OCH₂CH₃ | H | —CH₃ | —CH₃ | cyclopropyl- |
| | | | | | carbonyl |

Particular exemplary compounds according to the present invention may include, without being restricted thereto, any compound selected from

1-[2-(4-Hydroxy-3,5-dimethyl-phenyl)-8,9-dimethoxy-3-methyl-5,6-dihydro-pyrrolo[2,1-a]isoquinolin-1-yl]-propan-1-one,

1-[2-(2-Fluoro-3,4-dimethoxy-phenyl)-8,9-dimethoxy-3-methyl-5,6-dihydro-pyrrolo[2,1-a]isoquinolin-1-yl]-propan-1-one,

1-(2-Benzo[b]thiophen-3-yl-8,9-dimethoxy-3-methyl-5,6-dihydro-pyrrolo[2,1-a]isoquinolin-1-yl)-propan-1-one,

1-[2-(1H-Indol-3-yl)-8,9-dimethoxy-3-methyl-5,6-dihydro-pyrrolo[2,1-a]isoquinolin-1-yl]-propan-1-one,

1-(2-Biphenyl-4-yl-8,9-dimethoxy-3-methyl-5,6-dihydro-pyrrolo[2,1-a]isoquinolin-1-yl)-propan-1-one, and

1-Cyclopropyl-1-[2-(4-hydroxy-3,5-dimethyl-phenyl)-8,9-dimethoxy-3-methyl-5,6-dihydro-pyrrolo[2,1-a]isoquinolin-1-yl]-methanone,

and the salts, stereoisomers and the salts of the stereoisomers thereof.

A special interest within the present invention refers to those compounds according to this invention which are included, within the meaning of this invention, by one or, when possible, by a combination of more of the following special embodiments:

A special embodiment (embodiment 1) of the compounds according to the present invention refers to those compounds of formula I, in which

none of R1, R2 and R3 is bound to the 10-position of the pyrrolo[2.1-a]isoquinoline ring.

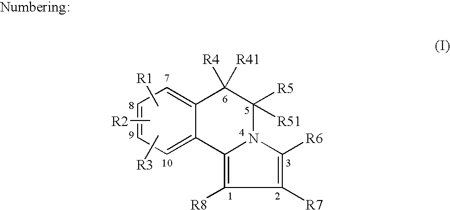

Another special embodiment (embodiment 2) of the compounds according to the present invention refers to those compounds of formula I, in which

none of R1 and R2 is bound to the 7- or 10-position of the pyrrolo[2.1-a]isoquinoline ring, and
R3 is hydrogen.

Another special embodiment (embodiment 3) of the compounds according to the present invention refers to those compounds of formula I, in which

either
R1 is bonded in the 8-position of the pyrrolo[2.1-a]isoquinoline ring, and is chlorine, 2-methoxy-ethoxy or difluoromethoxy, and
R2 is bonded in the 9-position of the pyrrolo[2.1-a]isoquinoline ring, and is 1-2C-alkoxy,

or
R1 is bonded in the 9-position of the pyrrolo[2.1-a]isoquinoline ring, and is chlorine, fluorine, nitro, methyl, amino or difluoromethoxy, and
R2 is bonded in the 8-position of the pyrrolo[2.1-a]isoquinoline ring, and is 1-2C-alkoxy,

and
R3 is hydrogen.

Another special embodiment (embodiment 4) of the compounds according to the present invention refers to those compounds of formula I, in which

either
R1 is bonded in the 8-position of the pyrrolo[2.1-a]isoquinoline ring, and is chlorine, 2-methoxy-ethoxy or difluoromethoxy, and
R2 is bonded in the 9-position of the pyrrolo[2.1-a]isoquinoline ring, and is 1-2C-alkoxy,

or
R1 is bonded in the 9-position of the pyrrolo[2.1-a]isoquinoline ring, and is chlorine, fluorine or difluoromethoxy, and
R2 is bonded in the 8-position of the pyrrolo[2.1-a]isoquinoline ring, and is 1-2C-alkoxy,

and
R3 is hydrogen.

Another special embodiment (embodiment 5) of the compounds according to the present invention refers to those compounds of formula I, in which

either
R1 is bonded in the 8-position of the pyrrolo[2.1-a]isoquinoline ring, and is 1-2C-alkoxy, such as e.g. methoxy or ethoxy, and
R2 is bonded in the 9-position of the pyrrolo[2.1-a]isoquinoline ring, and is 1-2C-alkoxy, such as e.g. methoxy or ethoxy,

and
R3 is hydrogen.

Another special embodiment (embodiment 6) of the compounds according to the present invention refers to those compounds of formula I, in which
R1 is bonded in the 8-position of the pyrrolo[2.1-a]isoquinoline ring, and is methoxy,
R2 is bonded in the 9-position of the pyrrolo[2.1-a]isoquinoline ring, and is methoxy, and
R3 is hydrogen.

Another special embodiment (embodiment 7) of the compounds according to the present invention refers to those compounds of formula I, in which
R1 is bonded in the 8-position of the pyrrolo[2.1-a]isoquinoline ring, and is ethoxy,
R2 is bonded in the 9-position of the pyrrolo[2.1-a]isoquinoline ring, and is methoxy, and
R3 is hydrogen.

Another special embodiment (embodiment 8) of the compounds according to the present invention refers to those compounds of formula I, in which
R1 is bonded in the 8-position of the pyrrolo[2.1-a]isoquinoline ring, and is difluoromethoxy,
R2 is bonded in the 9-position of the pyrrolo[2.1-a]isoquinoline ring, and is methoxy, and
R3 is hydrogen.

Another special embodiment (embodiment 9) of the compounds according to the present invention refers to those compounds of formula I, in which
R1 is bonded in the 9-position of the pyrrolo[2.1-a]isoquinoline ring, and is difluoromethoxy,
R2 is bonded in the 8-position of the pyrrolo[2.1-a]isoquinoline ring, and is methoxy, and
R3 is hydrogen.

Another special embodiment (embodiment 10) of the compounds according to the present invention refers to those compounds of formula I, in which
R1 is bonded in the 8-position of the pyrrolo[2.1-a]isoquinoline ring, and is 1-4C-alkoxy-2-4C-alkoxy, such as e.g. 2-methoxyethoxy,
R2 is bonded in the 9-position of the pyrrolo[2.1-a]isoquinoline ring, and is methoxy, and
R3 is hydrogen.

Another special embodiment (embodiment 11) of the compounds according to the present invention refers to those compounds of formula I, in which
R1 is bonded in the the 8-position of the pyrrolo[2.1-a]isoquinoline ring, and is halogen, such as e.g. fluorine or chlorine,
R2 is bonded in the 9-position of the pyrrolo[2.1-a]isoquinoline ring, and is methoxy, and
R3 is hydrogen.

Another special embodiment (embodiment 12) of the compounds according to the present invention refers to those compounds of formula I, in which
R1 is bonded in the the 9-position of the pyrrolo[2.1-a]isoquinoline ring, and is halogen, such as e.g. fluorine or chlorine,
R2 is bonded in the 8-position of the pyrrolo[2.1-a]isoquinoline ring, and is methoxy, and
R3 is hydrogen.

Another special embodiment (embodiment 13) of the compounds according to the present invention refers to those compounds of formula I, in which
R1 is bonded in the the 7-position of the pyrrolo[2.1-a]isoquinoline ring, and is halogen, such as e.g. fluorine or chlorine,
R2 is bonded in the 8-position of the pyrrolo[2.1-a]isoquinoline ring, and is methoxy, and
R3 is bonded in the 9-position of the pyrrolo[2.1-a]isoquinoline ring, and is methoxy.

Another special embodiment (embodiment 14) of the compounds according to the present invention refers to those compounds of formula I, in which
R1 is bonded in the the 7-position of the pyrrolo[2.1-a]isoquinoline ring, and is methoxy,
R2 is bonded in the 8-position of the pyrrolo[2.1-a]isoquinoline ring, and is methoxy, and
R3 is bonded in the 9-position of the pyrrolo[2.1-a]isoquinoline ring, and is methoxy.

Another special embodiment (embodiment 15) of the compounds according to the present invention refers to those compounds of formula I, in which
R4 is hydrogen, and
R41 is hydrogen.

Another special embodiment (embodiment 16) of the compounds according to the present invention refers to those compounds of formula I, in which
R4 is hydrogen or methyl, and
R41 is methyl.

Another special embodiment (embodiment 17) of the compounds according to the present invention refers to those compounds of formula I, in which
R6 is methyl.

Another special embodiment (embodiment 18) of the compounds according to the present invention refers to those compounds of formula I, in which
R7 is Har, or R74-substituted Har, in which
Har is a fused bicyclic 9- or 10-membered heteroaryl comprising a benzene ring and one or two heteroatoms, each of which is selected from a group consisting of nitrogen, oxygen and sulphur, such as e.g. quinolinyl, isoquinolinyl, indolyl, benzofuranyl or benzothiophenyl.

Another special embodiment (embodiment 19) of the compounds according to the present invention refers to those compounds of formula I, in which
R7 is naphthyl, quinolinyl, benzothiophenyl, or indolyl, such as e.g. naphthalen-1-yl, quinolin-4-yl, benzothiophen-3-yl or indol-3-yl.

Another special embodiment (embodiment 20) of the compounds according to the present invention refers to those compounds of formula I, in which
R7 is 3-dimethyamino-phenyl, 4-hydroxy-3,5-dimethylphenyl, or 1,1′-biphen-4-yl.

Another special embodiment (embodiment 21) of the compounds according to the present invention refers to those compounds of formula I, in which
R8 is ethylcarbonyl.

Another special embodiment (embodiment 22) of the compounds according to the present invention refers to those compounds of formula I, in which
R1 is bonded in the the 8-position of the pyrrolo[2.1-a]isoquinoline ring, and is 1-2C-alkoxy, such as e.g. methoxy,
R2 is bonded in the 9-position of the pyrrolo[2.1-a]isoquinoline ring, and is methoxy,
R3, R4, R41, R5, R51 are all hydrogen,
R6 is methyl, and
R8 is ethylcarbonyl.

Another special embodiment (embodiment 23) of the compounds according to the present invention refers to those compounds of formula I, in which
R1 is bonded in the the 8-position of the pyrrolo[2.1-a]isoquinoline ring, and is chlorine,
R2 is bonded in the 9-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is ethylcarbonyl.

Another special embodiment (embodiment 24) of the compounds according to the present invention refers to those compounds of formula I, in which
R1 is bonded in the the 8-position of the pyrrolo[2.1-a]isoquinoline ring, and is fluorine,
R2 is bonded in the 9-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is ethylcarbonyl.

Another special embodiment (embodiment 25) of the compounds according to the present invention refers to those compounds of formula I, in which
R1 is bonded in the the 8-position of the pyrrolo[2.1-a]isoquinoline ring, and is difluoromethoxy,
R2 is bonded in the 9-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is ethylcarbonyl.

Another special embodiment (embodiment 26) of the compounds according to the present invention refers to those compounds of formula I, in which
R1 is bonded in the the 8-position of the pyrrolo[2.1-a]isoquinoline ring, and is 2-methoxyethoxy,
R2 is bonded in the 9-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is ethylcarbonyl.

Another special embodiment (embodiment 27) of the compounds according to the present invention refers to those compounds of formula I, in which
R1 is bonded in the the 9-position of the pyrrolo[2.1-a]isoquinoline ring, and is 1-2C-alkoxy, such as e.g. methoxy,
R2 is bonded in the 8-position of the pyrrolo[2.1-a]isoquinoline ring, and is methoxy,
R3, R4, R41, R5, R51 are all hydrogen,
R6 is methyl, and
R8 is ethylcarbonyl.

Another special embodiment (embodiment 28) of the compounds according to the present invention refers to those compounds of formula I, in which
R1 is bonded in the the 9-position of the pyrrolo[2.1-a]isoquinoline ring, and is chlorine,
R2 is bonded in the 8-position of the pyrrolo[2.1-a]isoquinoline ring, and is 1-2-alkoxy, such as e.g. methoxy,
R3, R4, R41, R5, R51 are all hydrogen,
R6 is methyl, and
R8 is ethylcarbonyl.

Another special embodiment (embodiment 29) of the compounds according to the present invention refers to those compounds of formula I, in which
R1 is bonded in the the 9-position of the pyrrolo[2.1-a]isoquinoline ring, and is fluorine,
R2 is bonded in the 8-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is ethylcarbonyl.

Another special embodiment (embodiment 30) of the compounds according to the present invention refers to those compounds of formula I, in which
R1 is bonded in the the 9-position of the pyrrolo[2.1-a]isoquinoline ring, and is difluoromethoxy,
R2 is bonded in the 8-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is ethylcarbonyl.

Another special embodiment (embodiment 31) of the compounds according to the present invention refers to those compounds of formula I, in which
R1 is bonded in the the 9-position of the pyrrolo[2.1-a]isoquinoline ring, and is 2-methoxyethoxy,
R2 is bonded in the 8-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is ethylcarbonyl.

Another special embodiment (embodiment 32) of the compounds according to the present invention refers to those compounds of formula I, in which
R1 is bonded in the the 8-position of the pyrrolo[2.1-a]isoquinoline ring, and is 1-2C-alkoxy, such as e.g. methoxy,
R2 is bonded in the 9-position of the pyrrolo[2.1-a]isoquinoline ring, and is methoxy,
R3, R4, R41, R5, R51 are all hydrogen,
R6 is methyl, and
R8 is cyclopropylcarbonyl.

Another special embodiment (embodiment 33) of the compounds according to the present invention refers to those compounds of formula I, in which
R1 is bonded in the the 8-position of the pyrrolo[2.1-a]isoquinoline ring, and is chlorine,
R2 is bonded in the 9-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is cyclopropylcarbonyl.

Another special embodiment (embodiment 34) of the compounds according to the present invention refers to those compounds of formula I, in which
R1 is bonded in the the 8-position of the pyrrolo[2.1-a]isoquinoline ring, and is fluorine,
R2 is bonded in the 9-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is cyclopropylcarbonyl.

Another special embodiment (embodiment 35) of the compounds according to the present invention refers to those compounds of formula I, in which
R1 is bonded in the the 8-position of the pyrrolo[2.1-a]isoquinoline ring, and is difluoromethoxy,
R2 is bonded in the 9-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is cyclopropylcarbonyl.

Another special embodiment (embodiment 36) of the compounds according to the present invention refers to those compounds of formula I, in which
R1 is bonded in the the 8-position of the pyrrolo[2.1-a]isoquinoline ring, and is 2-methoxyethoxy,
R2 is bonded in the 9-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is cyclopropylcarbonyl.

Another special embodiment (embodiment 37) of the compounds according to the present invention refers to those compounds of formula I, in which
R1 is bonded in the the 9-position of the pyrrolo[2.1-a]isoquinoline ring, and is 1-2C-alkoxy, such as e.g. methoxy,
R2 is bonded in the 8-position of the pyrrolo[2.1-a]isoquinoline ring, and is methoxy,
R3, R4, R41, R5, R51 are all hydrogen,
R6 is methyl, and
R8 is cyclopropylcarbonyl.

Another special embodiment (embodiment 38) of the compounds according to the present invention refers to those compounds of formula I, in which
R1 is bonded in the the 9-position of the pyrrolo[2.1-a]isoquinoline ring, and is chlorine,
R2 is bonded in the 8-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is cyclopropylcarbonyl.

Another special embodiment (embodiment 39) of the compounds according to the present invention refers to those compounds of formula I, in which
R1 is bonded in the the 9-position of the pyrrolo[2.1-a]isoquinoline ring, and is fluorine,
R2 is bonded in the 8-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is cyclopropylcarbonyl.

Another special embodiment (embodiment 40) of the compounds according to the present invention refers to those compounds of formula I, in which
R1 is bonded in the the 9-position of the pyrrolo[2.1-a]isoquinoline ring, and is difluoromethoxy,
R2 is bonded in the 8-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is cyclopropylcarbonyl.

Another special embodiment (embodiment 41) of the compounds according to the present invention refers to those compounds of formula I, in which
R1 is bonded in the the 9-position of the pyrrolo[2.1-a]isoquinoline ring, and is 2-methoxyethoxy,
R2 is bonded in the 8-position of the pyrrolo[2.1-a]isoquinoline ring, and is 1-2C-alkoxy, such as e.g. methoxy,
R3, R4, R41, R5, R51 are all hydrogen,
R6 is methyl, and
R8 is cyclopropylcarbonyl.

Another special embodiment (embodiment 42) of the compounds according to the present invention refers to those compounds which are from formula Ia, in which
R5 and R51 are both hydrogen,
R6 is methyl, and
R1, R2, R3, R4 and R41 have any of the meanings indicated in Table 1 given above.

Another special embodiment (embodiment 43) of the compounds according to the present invention refers to those compounds which are from formula Ib, in which
R5 and R51 are both hydrogen,
R6 is methyl, and
R1, R2, R3, R4 and R41 have any of the meanings indicated in Table 1 given above.

Another special embodiment (embodiment 44) of the compounds according to the present invention refers to those compounds which are from formula Ia, in which
R5 and R51 are both hydrogen,
R6 is methyl, and
R1, R2, R3, R4 and R41 have any of the meanings indicated in Table 2 given above.

Another special embodiment (embodiment 45) of the compounds according to the present invention refers to those compounds which are from formula Ib, in which
R5 and R51 are both hydrogen,
R6 is methyl, and
R1, R2, R3, R4 and R41 have any of the meanings indicated in Table 2 given above.

Another special embodiment (embodiment 46) of the compounds according to the present invention refers to those compounds which are from formula Ia, in which
R5 and R51 are both hydrogen,
R6 is methyl, and
R1, R2, R3, R4, R41 and R8 have any of the meanings indicated in Table 1 given above.

Another special embodiment (embodiment 47) of the compounds according to the present invention refers to those compounds which are from formula Ib, in which
R5 and R51 are both hydrogen,
R6 is methyl, and
R1, R2, R3, R4, R41 and R8 have any of the meanings indicated in Table 1 given above.

Another special embodiment (embodiment 48) of the compounds according to the present invention refers to those compounds which are from formula Ia, in which
R5 and R51 are both hydrogen,
R6 is methyl, and
R1, R2, R3, R4, R41 and R8 have any of the meanings indicated in Table 2 given above.

Another special embodiment (embodiment 49) of the compounds according to the present invention refers to those compounds which are from formula Ib, in which
R5 and R51 are both hydrogen,
R6 is methyl, and
R1, R2, R3, R4, R41 and R8 have any of the meanings indicated in Table 2 given above.

It is to be understood that the present invention includes any or all possible combinations and subsets of the special embodiments defined hereinabove.

The compounds according to the present invention can be prepared, for example, in an art-known manner, or in a manner described and shown as follows, or as disclosed in WO 02/48144, WO 03/014115, WO 03/014116, WO,03/014117 or WO 03/051877 (the disclosure of which is incorporated herein), or as described by way of example in the following examples, or analogously or similarly thereto.

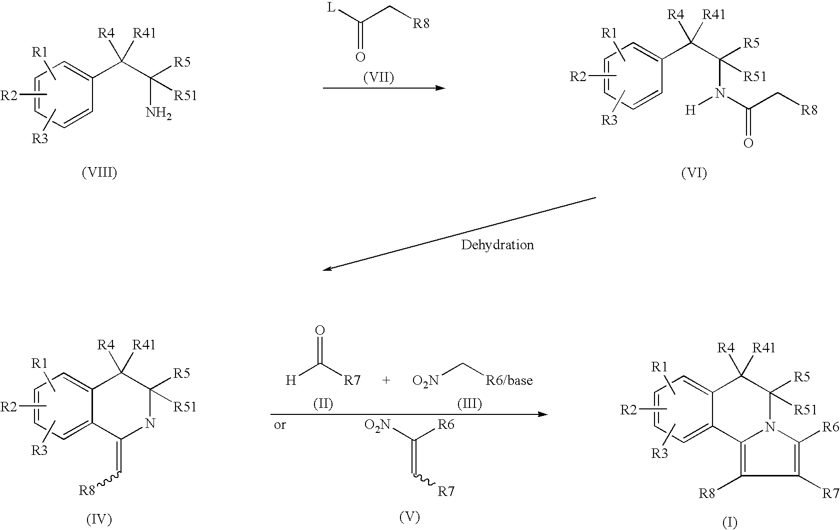

As shown in the scheme above, in a first reaction step compounds of formula VIII, in which R1, R2, R3, R4, R41, R5 and R51 have the meanings indicated above, are reacted with compounds of formula VII, in which R8 has the meanings indicated above and L is a suitable leaving group, for example chlorine or an acyloxy radical (e.g. the R8-CH₂—C(O)—O— radical), to give in the presence of a suitable organic or inorganic base corresponding compounds of formula VI.

Alternatively, compounds of formula VI are also accessible from compounds of formula VIII, in which R1, R2, R3, R4, R41, R5 and R51 have the meanings indicated above, and compounds of formula VII, in which R8 has the meanings indicated above and L is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. O-(benzotriazol-1-yl)-N,N,N′,N′-tetramethyluronium tetrafluoroborate or O-(benzotriazol-1yl)-N,N,N′,N′-tetramthyl-uronium-hexafluorophosphate] and N,N′-carbonyldiimidazole. In the scope of this invention preferred amide bond linking reagents are uronium salts and, particularly, carbodiimides, preferably, 1-ethyl-3-(3-dimethylaminopropyl)carbodiimide hydrochloride.

Said reactions are carried out under conditions known to the person skilled in the art or as described exemplarily in the following examples.

As shown in the next step, compounds of the formula IV, in which R1, R2, R3, R4, R41, R5, R51 and R8 have the meanings indicated above, can be obtained by cyclocondensation of corresponding compounds of the formula VI. Said cyclocondensation reaction is carried out in a manner habitual per se to the person skilled in the art or as described by way of example in the following examples, according to Bischler-Napieralski (e.g. as described in J. Chem. Soc., 1956, 4280-4282) in the presence of a suitable condensing or dehydrating agent, such as, for example, polyphosphoric acid, phosphorus pentachloride, phosphorus pentoxide or phosphorus oxychloride, in a suitable inert solvent, e.g. in a chlorinated hydrocarbon such as chloroform, or in a cyclic hydrocarbon such as toluene or xylene, or another inert solvent such as acetonitrile, or without further solvent using an excess of condensing agent, at reduced temperature, or at room temperature, or at elevated temperature or at the boiling temperature of the solvent or condensing agent used.

Compounds of formula IV are converted either with compounds of formulae II, in which R7 has the meanings given above, and III, in which R6 is 1-6C-alkyl or 1-4C-alkyl substituted by 1-4C-alkoxycarbonyl, or with compounds of formula V, in which R7 has the meanings given above and R6 is 1-6C-alkyl, or 1-4C-alkyl substituted by 1-4C-alkoxycarbonyl, optionally in a one pot synthesis and suitably in the presence of an inorganic or organic base (in particular a cyclic amine, e.g. piperidine) into the corresponding compounds of formula I.

Said conversion can be carried out as known to the skilled person or as described in the following examples or analogously or similarly thereto.

Compounds of formulae VIII, VII, III and II are commercially available or can be obtained in a manner described in the following examples or known to the skilled person from his/her expert knowledge and/or from literature, or analogously or similarly thereto.

Thus, e.g. compounds of formula VIII can be obtained starting from the corresponding benzaldehydes or acetophenons by a Henry reaction using nitromethane and subsequent reduction of the nitro group and the double bond in a manner customary per se to the skilled person (using e.g. LiAlH₄, see e.g. Zhurnal Organicheskoi Khimii, 1989, 25(7), 1477-82 or J. Org. Chem. 2005, 70(14), 5519-27), or in analogy to the sequence described in J. Med. Chem. 1987, 30(10), 1914-1918.

The mentioned benzaldehydes and acetophenons are known or can be obtained in analogy to known procedures or as described in the following examples.

Compounds of formula V are known or are accessible by reaction of compounds of formula II with compounds of formula III in the presence of a suitable organic or inorganic base in a manner customary per se to the skilled person.

Compounds of formula I obtained can be converted into further compounds of formula I by methods known to one of ordinary skill in the art. More specifically, for example, from compounds of the formula I, in which
a.) R61 or R71 or R74 are an ester group, the corresponding acids can be obtained by acidic or, particularly, alkaline hydrolysis.
b.) R6 is 1-4C-alkyl substituted by 1-4C-alkoxycarbonyl can be obtained by oxidation and esterification, e.g. from the appropriate 1-4C-alkyl substituted by hydroxyl, which can be obtained from 1-4C-alkyl by oxidation or from 1-4C-alkyl substituted by chlorine by hydroxylation (1-4C-alkyl substituted by chlorine can be obtained from 1-4C-alkyl by chlorination).

The method mentioned under a.) or b.) is expediently carried out analogously to the methods known to the person skilled in the art.

Optionally, compounds of the formula I can be converted into their salts, or, optionally, salts of the compounds of the formula I can be converted into the free compounds. Corresponding processes are habitual per se to the skilled person.

It is moreover known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in “Protective Groups in Organic Synthesis” by T. Greene and P. Wuts (John Wiley & Sons, Inc. 1999, 3^{rd} Ed.) or in “Protecting Groups (Thieme Foundations Organic Chemistry Series N Group” by P. Kocienski (Thieme Medical Publishers, 2000).

The isolation and purification of the substances according to the invention is carried out in a manner known per se, e.g. by distilling off the solvent in vacuo and recrystallizing the resulting residue from a suitable solvent or subjecting it to one of the customary purification methods, such as, for example, column chromatography on suitable support material.

Salts are obtained by dissolving the free compound in a suitable solvent (e.g. a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low molecular weight aliphatic alcohol such as methanol, ethanol or isopropanol) which contains the desired acid or base, or to which the desired acid or base is then added. The salts are obtained by filtering, reprecipitating, precipitating with a nonsolvent for the addition salt or by evaporating the solvent. Salts obtained can be converted by alkalization or by acidification into the free compounds, which in turn can be converted into salts. In this way, pharmacologically intolerable salts can be converted into pharmacologically tolerable salts.

Suitably, the conversions mentioned in this invention can be carried out analogously or similarly to methods which are familiar per se to the person skilled in the art.

The person skilled in the art knows on the basis of his/her knowledge and on the basis of those synthesis routes, which are shown and described within the description of this invention, how to find other possible synthesis routes for compounds of the formula I. All these other possible synthesis routes are also part of this invention.

The present invention also relates to intermediates and methods useful in synthesizing compounds according to this invention.

Having described the invention in detail, the scope of the present invention is not limited only to those described characteristics or embodiments. As will be apparent to persons skilled in the art, modifications, analogies, variations, derivations, homologisations and adaptations to the described invention can be made on the base of the disclosure (e.g. the explicite, implicite or inherent disclosure) of the present invention without departing from the spirit and scope of this invention as defined by the scope of the appended claims.

The following examples serve to illustrate the invention in greater detail without restricting it. Likewise, further compounds of the formula I, whose preparation is not explicitly described, can also be prepared in an analogous manner or in a manner familiar per se to the person skilled in the art using customary process techniques.

In the examples, m.p. stands for melting point, h for hour(s), min for minutes, conc. for concentrated, satd. for saturated, MS for mass spectrum, M for molecular ion, other abbreviations have their meanings customary per se to the skilled person.

Unless otherwise noted, when the exemplary compounds mentioned expressis verbis herein contain a chirality center, they are described illustratively as racemic mixtures herein, without restricting this invention thereto. Accordingly, the pure enantiomers and the salts thereof are also part of the invention.

The compounds of formula I mentioned in the examples, particularly which are mentioned as final compounds, and the stereoisomers, as well as the salts of these compounds and stereoisomers are a preferred subject of the invention.

### EXAMPLES

Final Products

### 1. 1-[2-(4-Hydroxy-3,5-dimethyl-phenyl)-8,9-dimethoxy-3-methyl-5,6-dihydro-pyrrolo[2,1-a]isoquinolin-1-yl]-propan-1-one

Analogously to a procedure described by Meyer in Liebigs Ann. Chem. 1981, 9, 1534-1544, 1-(6,7-dimethoxy-3,4-dihydro-2H-isoquinolin-1-ylidene)-butan-2-one (compound Al) is reacted with nitro ethane and 4-hydroxy-3,5-dimethyl benzaldehyde to afford the title compound:

A mixture of 150 mg (573 μmol) 1-(6,7-dimethoxy-3,4-dihydro-2H-isoquinolin-1-ylidene)-butan-2-one (compound A1), 172 mg (1.14 mmol) 4-hydroxy-3,5-dimethyl benzaldehyde, 82 μl (1.14 mmol) nitroethane and 28 μl (286 μmol) pyridine in a mixture of 2 ml ethanol and 2 ml 2-propanol is stirred at 70° C. for 20 h. The solvents are removed at reduced pressure. The residue is washed with hot 2-propanol. 130 mg of the title compound are obtained as pale yellow crystals. M.p.: 188-190° C. The mass spectrum shows the molecular peak M+H at 420.0 Da.

The following examples (Examples 2-5) can be prepared in analogy to example 1 using 1-(6,7-dimethoxy-3,4-dihydro-2H-isoquinolin-1-ylidene)-butan-2-one (compound A1) as starting compound. All aldehydes used are commercially available or can be prepared in analogy to published procedures. If nitro propane or 4-nitro butyric acid methyl ester is used instead of nitroethane, 3-ethyl-5,6-dihydro-pyrrolo[2, 1-a]isoquinolines and 3-(8,9-dimethoxy-5,6-dihydro-pyrrolo[2,1-a]isoquinolin-3-yl)propionic methyl esters, respectively can be obtained.

### 2. 1-[2-(2-Fluoro-3,4-dimethoxy-phenyl)-8,9-dimethoxy-3-methyl-5,6-dihydro-pyrrolo[2,1-a]isoquinolin-1-yl]-propan-1-one

M.p.: 129-132° C. The mass spectrum shows the molecular peak M+H at 454.1 Da

### 3. 1-(2-Benzo[b]thiophen-3-yl-8,9-dimethoxy-3-methyl-5,6-dihydro-pyrrolo[2,1-a]isoquinolin-1-yl)-propan-1-one

M.p.: 181-183° C. The mass spectrum shows the molecular peak M+H at 432.1 Da

### 4. 1-[2-(1H-Indol-3-yl)-8,9-dimethoxy-3-methyl-5,6-dihydro-pyrrolo[2,1-a]isoquinolin-1-yl]-propan-1-one

M.p.: 238-240° C. The mass spectrum shows the molecular peak M+H at 415.2 Da

### 5. 1-(2-Biphenyl-4-yl-8,9-dimethoxy-3-methyl-5,6-dihydro-pyrrolo[2,1-a]isoquinolin-1-yl)-propan-1-one

M.p.: 209-211° C. The mass spectrum shows the molecular peak M+H at 452.2 Da

### 6. 1-Cyclopropyl-1-[2-(4-hydroxy-3,5-dimethyl-phenyl)-8,9-dimethoxy-3-methyl-5,6-dihydro-pyrrolo[2,1-a]isoquinolin-1-yl]-methanone

Analogously to a procedure described by Meyer in Liebigs Ann. Chem. 1981, 9, 1534-1544,1-cyclopropyl-2-(6,7-dimethoxy-3,4-dihydro-2H-isoquinolin-1-ylidene)-ethanone (compound A2) is reacted with nitro ethane and 4-hydroxy-3,5-dimethyl benzaldehyde to afford the title compound. M.p.: decomposition. The mass spectrum shows the molecular peak M+H at 432.0 Da.

Starting Compounds

### A1. 1-(6,7-Dimethoxy-3,4-dihydro-2H-isoquinolin-1-ylidene)-butan-2-one

A solution of 700 mg (2.50 mmol) 3-oxo-pentanoic acid [2-(3,4-dimethoxy-phenyl)-ethyl]-amide (compound B1) in 12 ml toluene is heated to reflux and 2.48 g (17.5 mmol) P₂O₅ are added in one portion. After heating to reflux for 15 min the solution is cooled to room temperature. Ice is added. After warming up to room temperature potassium carbonate is added until the solution is alkaline and. The mixture is extracted with ethyl acetate and the organic layer is dried with magnesium sulfate. After column chromatography 150 mg of the title compound are obtained as a pale yellow solid.

### A2. 1-Cyclopropyl-2-(6,7-dimethoxy-3,4-dihydro-2H-isoquinolin-1-ylidene)-ethanone

The title compound can be obtained by a Bischler-Napieralski reaction (e.g. Ber. 1893, 26, 1903) using 3-cyclopropyl-N-[2-(3,4-dimethoxy-phenyl)-ethyl]-3-oxo-propionamide (compound B2) as the starting material or analogously as described for compound A1.

### B1. 3-Oxo-pentanoic acid[2-(3,4-dimethoxy-phenyl)-ethyl]-amide

To a solution of 2.68 ml (15.9 mmol) 2-(3,4-dimethoxy-phenyl)-ethylamine (compoundC1) in 10 ml toluene at 0° C. are added 9.95 ml (19.9 mmol) AlMe₃ (2 M solution in toluene). The ice bath is removed and the solution is allowed to warm up to room temperature. A solution of 1 ml (7.96 mmol) 3-oxo-pentanoic acid methyl ester in 10 ml toluene is added to the reaction mixture. The solution is heated to 80° C. for 20 h. After cooling to room temperature an aqueous solution of sodium hydroxide is added until the pH is basic. The mixture is extracted with ethyl acetate and the organic layer is dried with magnesium sulfate. The solvent is removed at reduced pressure and the residue is purified by column chromatography. 700 mg of the title compound are obtained as a colourless oil.

### B2. 3-Cyclopropyl-N-[2-(3,4-dimethoxy-phenyl)-ethyl]-3-oxo-propionamide

The title compound can be prepared by reaction of 2-(3,4-dimethoxy-phenyl)-ethylamine (compound C1) with 3-cyclopropyl-3-oxo-propionic acid methyl ester in analogy to compound B1.

### C1. 2-(3,4-Dimethoxy-phenyl)-ethylamine

The title compound is commercially available.

The appropriate starting compounds for the preparation of further compounds are commercially available, or can be prepared as described below in the synthesis of the compounds C2 to C4 or analogously or similarly thereto, or can be obtained in analogy to published procedures, e.g. the substituted 2-phenethyl-amines can be prepared starting from the corresponding benzaldehydes by standard procedures (see also Shepard et al., J. Org. Chem. 1952, 17, 568).

### C2. 2-[4-Methoxy-3-(2-methoxy-ethoxy)-phenyl)-ethylamine

2-[4-Methoxy-3-(2-methoxy-ethoxy)-phenyl)-ethylamine can be prepared by alkylation of 4-methoxy-3-hydroxy benzaldehyde with 2-bromomethyl ethyl ether (analogous to a procedure by Ashton et al., J. Med. Chem. 1994, 37, 1696-1703), followed by a sequence described by Shepard et al. in J. Org. Chem. 1952, 17, 568.

MS (M+H)=226.0

### C3. 2-[4-(1,1-Difluoro-methoxy)-3-methoxy-phenyl]-ethylamine

2-[4-(1,1-Difluoro-methoxy)-3-methoxy-phenyl]-ethylamine can be prepared by difluoromethylation of 4-hydroxy-3-methoxy benzaldehyde with chloro difluoro methane according to a procedure published by Amschler et al. (WO97/28131), followed by a sequence described by Shepard et al. in J. Org. Chem. 1952, 17, 568.

MS (M+H)=217.6

### C4. 2-[3-(1,1-Difluoro-methoxy)-4-methoxy-phenyl]-ethylamine

2-[3-(1,1-Difluoro-methoxy)-4-methoxy-phenyl]-ethylamine can be prepared by difluoromethylation of 3-hydroxy-4-methoxy benzaldehyde with chloro difluoro methane according to a procedure published by Amschler et al. (WO97/28131), followed by a sequence described by Shepard et al. in J. Org. Chem. 1952, 17, 568.

MS (M+H)=217.7

### C5. (RS)-2-(3,4-Dimethoxy-phenyl)-2-methyl-ethylamine

The title compound can be prepared starting from the corresponding acetophenone derivative by standard procedures, e.g. in analogy to a sequence described by Shepard et al. in J. Org. Chem. 1952, 17, 568, or in J. Med. Chem. 1987, 30(10), 1914-1918.

Commercial Utility

The compounds according to the invention have miscellaneous valuable pharmacological properties which make them commercially utilizable.

The compounds according to the invention therefore can be employed as therapeutic agents for the treatment and prophylaxis of diseases in human and veterinary medicine.

Thus, for example, in more embodimental detail, the compounds according to this invention are potent and highly efficacious inhibitors of cellular (hyper)proliferation and/or inducers of apoptosis in cancer cells. Therefore, these compounds are expected to be useful for treating (hyper)proliferative diseases and/or disorders responsive to the induction of apoptosis, in particular cancer.

Further on, these compounds can be useful in the treatment of benign or malignant neoplasia. A “neoplasia” is defined by cells displaying aberrant cell proliferation and/or survival and/or a block in differentiation. A “benign neoplasia” is described by hyperproliferation of cells, incapable of forming an aggressive, metastasizing tumor in-vivo. In contrast, a “malignant neoplasia” is described by cells with multiple cellular and biochemical abnormalities, capable of forming a systemic disease, for example forming tumor metastasis in distant organs.

Various diseases are caused by limitless replicative potential and aberrant cell proliferation (“hyperproliferation”) as well as evasion from apoptosis. These diseases include e.g. benign hypoplasia like that of the prostate (“BPH”) or colon epithelium, psoriasias, glomerulonephritis or osteoarthritis. Most importantly these diseases include malignant neoplasia commonly described as cancer and characterized by tumor cells finally metastasizing into distinct organs or tissues. Malignant neoplasia include solid and hematological tumors. Solid tumors are exemplified by tumors of the breast, bladder, bone, brain, central and peripheral nervous system, colon, endocrine glands (e.g. thyroid and adrenal cortex), esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva. Malignant neoplasia include inherited cancers exemplified by retinoblastoma and Wilms tumor. In addition, malignant neoplasia include primary tumors in said organs and corresponding secondary tumors in distant organs (“tumor metastases”). Hematological tumors are exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, cancers of unknown primary site as well as AIDS related malignancies.

It is to be noted that a cancer disease as well as a malignant neoplasia does not necessarily require the formation of metastases in distant organs. Certain tumors exert devastating effects on the primary organ itself through their aggressive growth properties. These can lead to the destruction of the tissue and organ structure finally resulting in failure of the assigned organ function.

Neoplastic cell proliferation might effect normal cell behaviour and organ function. For example the formation of new blood vessels, a process described as neovascularization, is induced by tumors or tumor metastases. Compounds according to this invention can be commercially applicable for treatment of pathophysiological relevant processes caused by benign or neoplastic cell proliferation, such as but not limited to neovascularization by unphysiological proliferation of vascular endothelial cells.

Drug resistance is of particular importance for the frequent failure of standard cancer therapeutics. This drug resistance is caused by various cellular and molecular mechanisms like overexpression of drug efflux pumps or mutation within the cellular target protein. The commercial applicability of the compounds according to this invention is not limited to 1^{st} line treatment of patients. Patients with resistance to defined cancer chemotherapeutics or target specific anti-cancer drugs (2^{nd} or 3^{rd} line treatment) can be also amenable for treatment with the compounds according to this invention.

Further, the compounds according to this invention are found to be cell-cycle specific, e.g. they induce apoptosis particularly in continously proliferating cells actively passing the S-phase (“DNA synthesis”) of the cell cycle, but not in resting, non-dividing cells.

Thus, the compounds of the present invention are expected to be highly efficacious in anti-proliferative therapy and to have a higher therapeutic index compared to standard chemotherapeutic drugs targeting cells irrespective of their proliferative status (e.g. cisplatin, doxorubicin).

In another facet of the present invention, the compounds according to this invention show interesting properties, which may make them useful in the therapy of T-cell associated diseases, for suppression of the immune system, for treating restenosis and/or, if appropriate, for modulating angiogenesis.

Further on, a special interest in the compounds according to the present invention lies in their potency to combat (hyper)proliferative diseases and/or disorders responsive to the induction of apoptosis, in particular cancer, independently from or uncorrelated with their PDE10 inhibitory capacity.

Compounds according to the present invention can be commercially applicable for treatment, prevention or amelioration of the diseases of benign and malignant behavior as described before, such as e.g. benign or malignant neoplasia, particularly cancer, such as e.g. any of those cancer diseases described above.

In the context of their properties, functions and usabilities mentioned herein, the compounds according to the present invention are expected to be distinguished by valuable and desirable effects related therewith, such as e.g. by low toxicity, superior bioavailability in general (such as e.g. good enteral absorption), superior therapeutic window, absence of significant side effects, and/or further beneficial effects related with their therapeutic and pharmaceutical suitability.

The invention further includes a method for treating (hyper)proliferative diseases and/or disorders responsive to the induction of apoptosis, particularly those diseases, disorders, conditions or illnesses mentioned above, in mammals, including humans, suffering therefrom comprising administering to said mammals in need thereof a pharmacologically active and therapeutically effective and tolerable amount of one or more of the compounds according to this invention.

The present invention further includes a method useful to modulate apoptosis and/or aberrant cell growth in the therapy of benign or malignant neoplastic diseases, such as e.g. cancer, comprising administering to a subject in need of such therapy a therapeutically active and pharmacologically effective and tolerable amount of one or more of the compounds according to this invention.

The present invention further relates to the use of the compounds according to this invention for the production of pharmaceutical compositions which are employed for the treatment, prophylaxis and/or amelioration of the illnesses mentioned.

The present invention further relates to the use of the compounds according to this invention for the production of pharmaceutical compositions which can be used in the treatment, prevention or amelioration of (hyper)proliferative diseases of benign or malignant behaviour and/or disorders responsive to the induction of apoptosis in a mammal, such as, for example, benign or malignant neoplasia, e.g. cancer.

The present invention further relates to the use of the compounds according to this invention for the production of pharmaceutical compositions which can be used use in the treatment, prevention or amelioration of disorders responsive to arresting of aberrant cell growth and/or induction of apoptosis.

The present invention further relates to the use of the compounds according to this invention for the production of pharmaceutical compositions for treating, preventing or ameliorating benign or malignant neoplasia, particularly cancer, such as e.g. any of those cancer diseases described above.

The present invention further relates to pharmaceutical compositions comprising one or more of the compounds according to this invention and a pharmaceutically acceptable carrier or diluent.

The present invention further relates to pharmaceutical compositions made by combining one or more of the compounds according to this invention and a pharmaceutically acceptable carrier or diluent.

The present invention further relates to a combination comprising a compound according to this invention and a pharmaceutically acceptable excipient, carrier and/or diluent, e.g. for treating, preventing or ameliorating benign or malignant neoplasia, particularly cancer, such as e.g. any of those cancer diseases described above.

The present invention further relates to a composition consisting essentially of a therapeutically effective and tolerable amount of one or more compounds according to this invention together with the usual pharmaceutically acceptable vehicles, diluents and/or excipients for use in therapy, e.g. for treating, preventing or ameliorating hyperproliferative diseases, such as e.g. cancer, and/or disorders responsive to induction of apoptosis.

The present invention further relates to compounds according to this invention for use in therapy, such as, for example, in the treatment, prevention or amelioration of (hyper)proliferative diseases of benign or malignant behaviour and/or disorders responsive to the induction of apoptosis, such as e.g. those diseases mentioned herein, particularly cancer.

The present invention further relates to compounds according to this invention having anti-proliferative and/or apoptosis inducing activity.

The present invention further relates to pharmaceutical compositions according to this invention having anti-proliferative activity.

The present invention further relates to pharmaceutical compositions according to this invention having apoptosis inducing activity.

The invention further relates to the use of a pharmaceutical composition comprising one or more of the compounds according to this invention as sole active ingredient(s) and a pharmaceutically acceptable carrier or diluent in the manufacture of pharmaceutical products for the treatment and/or prophylaxis of the illnesses mentioned above.

Additionally, the invention relates to an article of manufacture, which comprises packaging material and a pharmaceutical agent contained within said packaging material, wherein the pharmaceutical agent is therapeutically effective inhibiting cellular (hyper)proliferation and/or inducing apoptosis, ameliorating the symptoms of a (hyper)proliferative disease and/or a disorder responsive to the induction of apoptosis, and wherein the packaging material comprises a label or package insert which indicates that the pharmaceutical agent is useful for treating, preventing or ameliorating a (hyper)proliferative disease and/or a disorder responsive to the induction of apoptosis, and wherein said pharmaceutical agent comprises one or more compounds according to the invention. The packaging material, label and package insert otherwise parallel or resemble what is generally regarded as standard packaging material, labels and package inserts for pharmaceuticals having related utilities.

The pharmaceutical compositions according to this invention are prepared by processes which are known per se and familiar to the person skilled in the art. As pharmaceutical compositions, the compounds of the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other active compound excipients, for example antioxidants, dispersants, emulsifiers, pre-servatives, solubilizers, colorants, complexing agents or permeation promoters, can be used.

The administration of the compounds, pharmaceutical compositions or combinations according to the invention may be performed in any of the generally accepted modes of administration available in the art. Illustrative examples of suitable modes of administration include intravenous, oral, nasal, parenteral, topical, transdermal and rectal delivery. Oral and intravenous delivery are preferred.

For the treatment of dermatoses, the compounds of the invention can be in particular administered in the form of those pharmaceutical compositions which are suitable for topical application. For the production of the pharmaceutical compositions, the compounds of the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the compounds of the invention (=active compounds) is carried out in the order of magnitude customary for inhibitors of cellular (hyper)proliferation or apoptosis inducers. Topical application forms (such as ointments) for the treatment of dermatoses thus contain the active compounds in a concentration of, for example, 0.1-99%. The customary dose in the case of systemic therapy (p.o.) may be between 0.03 and 60 mg/kg per day, (i. v.) may be between 0.03 and 60 mg/kg/h. In another embodiment, the customary dose in the case of systemic therapy (p.o.) is between 0.3 and 30 mg/kg per day, (i. v.) is between 0.3 and 30 mg/kg/h.

The choice of the optimal dosage regime and duration of medication, particularly the optimal dose and manner of administration of the active compounds necessary in each case can be determined by a person skilled in the art on the basis of his/her expert knowledge.

Furthermore, the compounds of the present invention show interesting and surprising properties, which may make them particular useful in combination therapy, particularly of those diseases described herein.

Thus, for example, the compounds according to the present invention can act synergistically with other active agents, that may be beneficial in the therapy of those diseases mentioned herein, particularly cancer.

Depending upon the particular disease, to be treated or prevented, additional therapeutic active agents, which are normally administered to treat or prevent that disease, may optionally be coadministered with the compounds according to this invention. As used herein, additional therapeutic agents that are normally administered to treat or prevent a particular disease are known as appropriate for the disease being treated.

For example, compounds according to this invention may be combined with one or more standard therapeutic agents used for treatment of the diseases as mentioned before.

In one particular embodiment, compounds according to this invention may be combined with one or more art-known anti-cancer agents, such as e.g. with one or more chemotherapeutic and/or target specific anti-cancer agents as described below.

Examples of known chemotherapeutic anti-cancer agents frequently used in combination therapy include, but not are limited to (i) alkylating/carbamylating agents such as Cyclophosphamid (Endoxan®), Ifosfamid (Holoxan®), Thiotepa (Thiotepa Lederle®), Melphalan (Alkeran®), or chloroethylnitrosourea (BCNU); (ii) platinum derivatives like cis-platin (Platinex® BMS), oxaliplatin or carboplatin (Cabroplat® BMS); (iii) antimitotic agents/tubulin inhibitors such as vinca alkaloids (vincristine, vinblastine, vinorelbine), taxanes such as Paclitaxel (Taxol®), Docetaxel (Taxotere®) and analogs as well as new formulations and conjugates thereof, epothilones such as Epothilone B (Patupilone®), Azaepothilone (Ixabepilone®) or ZK-EPO, a fully synthetic epothilone B analog; (iv) topoisomerase inhibitors such as anthracyclines (exemplified by Doxorubicin/Adriblastin®), epipodophyllotoxines (examplified by Etoposide/Etopophos®) and camptothecin and camptothecin analogs (exemplified by Irinotecan/Camptosar® or Topotecan/Hycamtin®); (v) pyrimidine antagonists such as 5-fluorouracil (5-FU), Capecitabine (Xeloda®), Arabinosylcytosine/Cytarabin (Alexan®) or Gemcitabine (Gemzar®); (vi) purin antagonists such as 6-mercaptopurine (Puri-Nethol®), 6-thioguanine or fludarabine (Fludara®) and finally (vii) folic acid antagonists such as methotrexate (Farmitrexat®) or premetrexed (Alimta®).

Examples of target specific anti-cancer drug classes used in experimental or standard cancer therapy include but are not limited to (i) kinase inhibitors such as e.g. Imatnib (Glivec®), ZD-1839/Gefitinib (Iressa®), Bay43-9006 (Sorafenib), SU11248/Sunitinib (Sutent®) or OSI-774/Erlotinib (Tarceva®); (ii) proteasome inhibitors such as PS-341/Bortezumib (Velcade®); (iii) histone deacetylase inhibitors like SAHA, PXD101, MS275, MGCD0103, Depsipeptide/FK228, NVP-LBH589, NVP-LAQ824, Valproic acid (VPA) and butyrates (iv) heat shock protein 90 inhibitors like 17-allylaminogeldanamycin (17-MG); (v) vascular targeting agents (VTAs) like combretastin A4 phosphate or AVE8062/AC7700 and anti-angiogenic drugs like the VEGF antibodies, such as Bevacizumab (Avastin®), or KDR tyrosine kinase inhibitors such as PTK787/ZK222584 (Vatalanib); (vi) monoclonal antibodies such as Trastuzumab (Herceptin®) or Rituximab (MabThera/Rituxan®) or Alemtuzumab (Campath®) or Tositumab (Bexxar®) or C225/Cetuximab (Erbitux®) or Avastin (see above) as well as mutants and conjugates of monoclonal antibodies, e.g. Gemtuzumab ozogamicin (Mylotarg®) or Ibritumomab tiuxetan (Zevalin®), and antibody fragments; (vii) oligonucleotide based therapeutics like G-3139/Oblimersen (Genasense®); (viii) Toll-like receptor/TLR 9 agonists like Promune®; (ix) protease inhibitors (x) hormonal therapeutics such as anti-estrogens (e.g. Tamoxifen or Raloxifen), anti-androgens (e.g. Flutamide or Casodex), LHRH analogs (e.g. Leuprolide, Goserelin or Triptorelin) and aromatase inhibitors.

Other known target specific anti-cancer agents which may be used for combination therapy include bleomycin, retinoids such as all-trans retinoic acid (ATRA), DNA methyltransferase inhibitors such as the 2-deoxycytidine derivative Decitabine (Docagen®) and 5-Azacytidine, alanosine, cytokines such as interleukin-2, interferons such as interferon α2 or interferon-γ, death receptor agonists, such as TRAIL, DR4/5 agonistic antibodies, FasL and TNF-R agonists.

As exemplary anti-cancer agents, which may be useful in the combination therapy according to the present invention, any of the following drugs may be mentioned, without being restricted thereto, 5 FU, actinomycin D, ABARELIX, ABCIXIMAB, ACLARUBICIN, ADAPALENE, ALEMTUZUMAB, ALTRETAMINE, AMINOGLUTETHIMIDE, AMIPRILOSE, AMRUBICIN, ANASTROZOLE, ANCITABINE, ARTEMISININ, AZATHIOPRINE, BASILIXIMAB, BENDAMUSTINE, BEVACIZUMAB, BEXXAR, BICALUTAMIDE, BLEOMYCIN, BORTEZOMIB, BROXURIDINE, BUSULFAN, CAMPATH, CAPECITABINE, CARBOPLATIN, CARBOQUONE, CARMUSTINE, CETRORELIX, CHLORAM-BUCIL, CHLORMETHINE, CISPLATIN, CLADRIBINE, CLOMIFENE, CYCLOPHOSPHAMIDE, DACARBAZINE, DACLIZUMAB, DACTINOMYCIN, DAUNORUBICIN, DECITABINE, DESLORELIN, DEXRAZOXANE, DOCETAXEL, DOXIFLURIDINE, DOXORUBICIN, DROLOXIFENE, DROSTANOLONE, EDELFOSINE, EFLORNITHINE, EMITEFUR, EPIRUBICIN, EPITIOSTANOL, EPTAPLATIN, ERBITUX, ERLOTINIB, ESTRAMUSTINE, ETOPOSIDE, EXEMESTANE, FADROZOLE, FINASTERIDE, FLOXURIDINE, FLUCYTOSINE, FLUDARABINE, FLUOROURACIL, FLUTAMIDE, FORMESTANE, FOSCARNET, FOSFESTROL, FOTEMUSTINE, FULVESTRANT, GEFITINIB, GENASENSE, GEMCITABINE, GLIVEC, GOSERELIN, GUSPERIMUS, HERCEPTIN, IDARUBICIN, IDOXURIDINE, IFOSFAMIDE, IMATINIB, IMPROSULFAN, INFLIXIMAB, IRINOTECAN, IXABEPILONE, LANREOTIDE, LETROZOLE, LEUPRORELIN, LOBAPLATIN, LOMUSTINE, LUPROLIDE, MELPHALAN, MERCAPTOPURINE, METHOTREXATE, METUREDEPA, MIBOPLATIN, MIFEPRISTONE, MILTEFOSINE, MIRIMOSTIM, MITOGUAZONE, MITOLACTOL, MITOMYCIN, MITOXANTRONE, MIZORIBINE, MOTEXAFIN, MYLOTARG, NARTOGRASTIM, NEBAZUMAB, NEDAPLATIN, NILUTAMIDE, NIMUSTINE, OCTREOTIDE, ORMELOXIFENE, OXALI-PLATIN, PACLITAXEL, PALIVIZUMAB, PATUPILONE, PEGASPARGASE, PEGFILGRASTIM, PEMETREXED, PENTETREOTIDE, PENTOSTATIN, PERFOSFAMIDE, PIPOSULFAN, PIRARUBICIN, PLICAMYCIN, PREDNIMUSTINE, PROCARBAZINE, PROPAGERMANIUM, PROSPIDIUM CHLORIDE, RALOXIFEN, RALTITREXED, RANIMUSTINE, RANPIRNASE, RASBURICASE, RAZOXANE, RITUXIMAB, RIFAMPICIN, RITROSULFAN, ROMURTIDE, RUBOXISTAURIN, SARGRAMOSTIM, SATRAPLATIN, SIROLIMUS, SOBUZOXANE, SORAFENIB, SPIROMUSTINE, STREPTOZOCIN, SUNITINIB, TAMOXIFEN, TASONERMIN, TEGAFUR, TEMOPORFIN, TEMOZOLOMIDE, TENIPOSIDE, TESTOLACTONE, THIOTEPA, THYMALFASIN, TIAMIPRINE, TOPOTECAN, TOREMIFENE, TRAIL, TRASTUZUMAB, TREOSULFAN, TRIAZIQUONE, TRIMETREXATE, TRIPTORELIN, TROFOSFAMIDE, UREDEPA, VALRUBICIN, VATALANIB, VERTEPORFIN, VINBLASTINE, VINCRISTINE, VINDESINE, VINORELBINE, VOROZOLE and ZEVALIN.

The anti-cancer agents mentioned herein above as combination partners of the compounds according to this invention are meant to include pharmaceutically acceptable derivatives thereof, such as e.g. their pharmaceutically acceptable salts.

In addition, compounds according to this invention may be combined with agents that interfere with cyclic nucleotide metabolism, such as e.g. phosphodiesterase inhibitors, protein kinase A or protein kinase G agonists or antagonists, activators or inhibitors of exchange protein activated by cyclic AMP (Epac) or cAMP-GEF or activators or inhibitors of guanylate cyclase or adenylate cyclase.

The person skilled in the art is aware on the base of his/her expert knowledge of the kind, total daily dosage(s) and administration form(s) of the additional therapeutic agent(s) coadministered. Said total daily dosage(s) can vary within a wide range.

In practicing the present invention, the compounds according to this invention may be administered in combination therapy separately, sequentially, simultaneously, concurrently or chronologically staggered (such as e.g. as combined unit dosage forms, as separate unit dosage forms, as adjacent discrete unit dosage forms, as fixed or non-fixed combinations, as kit-of-parts or as admixtures) with one or more standard therapeutics, in particular art-known ant-cancer agents (chemotherapeutic and/or target specific anti-cancer agents), such as e.g. any of those mentioned above.

In this context, the present invention further relates to a combination comprising

a first active ingredient, which is at least one compound according to this invention, and

a second active ingredient, which is at least one art-known anti-cancer agent, such as e.g. one or more of those mentioned herein above,

for separate, sequential, simultaneous, concurrent or chronologically staggered use in therapy, such as e.g. in therapy of any of those diseases mentioned herein.

The term “combination” according to this invention may be present as a fixed combination, a non-fixed combination or a kit-of-parts.

A “fixed combination” is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously, concurrently or chronologically staggered.

The present invention further relates to a pharmaceutical composition comprising

a first active ingredient, which is at least one compound according to this invention, and

a second active ingredient, which is at least one art-known anti-cancer agent, such as e.g. one or more of those mentioned herein above, and, optionally,

a pharmaceutically acceptable carrier or diluent,

for separate, sequential, simultaneous, concurrent or chronologically staggered use in therapy.

The present invention further relates to a combination product comprising

a.) at least one compound according to this invention formulated with a pharmaceutically acceptable carrier or diluent, and

b.) at least one art-known anti-cancer agent, such as e.g. one or more of those mentioned herein above, formulated with a pharmaceutically acceptable carrier or diluent.

The present invention further relates to a kit-of-parts comprising a preparation of a first active ingredient, which is a compound according to this invention, and a pharmaceutically acceptable carrier or diluent; a preparation of a second active ingredient, which is an art-known anti-cancer agent, such as one of those mentioned above, and a pharmaceutically acceptable carrier or diluent; for simultaneous, concurrent, sequential, separate or chronologically staggered use in therapy. Optionally, said kit comprises instructions for its use in therapy, e.g. to treat (hyper)proliferative diseases and/or disorders responsive to the induction of apoptosis, such as e.g. cancer, more precisely, any of those cancer diseases described above.

The present invention further relates to a combined preparation comprising at least one compound according to this invention and at least one art-known anti-cancer agent for simultaneous, concurrent, sequential or separate administration.

In this connection, the present invention further relates to combinations, compositions, formulations, preparations or kits according to the present invention having anti-proliferative and/or apoptosis inducing properties.

In addition, the present invention further relates to a method for treating in combination therapy (hyper)proliferative diseases and/or disorders responsive to the induction of apoptosis, such as e.g. cancer, in a patient comprising administering a combination, composition, formulation, preparation or kit as described herein to said patient in need thereof.

In addition, the present invention further relates to a method for treating (hyper)proliferative diseases of benign or malignant behaviour and/or disorders responsive to the induction of apoptosis, such as e.g. cancer, in a patient comprising administering in combination therapy separately, simultaneously, concurrently, sequentially or chronologically staggered a pharmaceutically active and therapeutically effective and tolerable amount of a pharmaceutical composition, which comprises a compound according to this invention and a pharmaceutically acceptable carrier or diluent, and a pharmaceutically active and therapeutically effective and tolerable amount of one or more art-known anti-cancer agents, such as e.g. one or more of those mentioned herein, to said patient in need thereof.

In further addition, the present invention relates to a method for treating, preventing or ameliorating (hyper)proliferative diseases and/or disorders responsive to induction of apoptosis, such as e.g. benign or malignant neoplasia, e.g. cancer, particularly any of those cancer diseases mentioned herein, in a patient comprising administering separately, simultaneously, concurrently, sequentially or chronologically staggered to said patient in need thereof an amount of a first active compound, which is a compound according to the present invention, and an amount of at least one second active compound, said at least one second active compound being a standard therapeutic agent, particularly at least one art-known anti-cancer agent, such as e.g. one or more of those chemotherapeutic and target-specific anti-cancer agents mentioned herein, wherein the amounts of the first active compound and said second active compound result in a therapeutic effect.

In yet further addition, the present invention relates to a method for treating, preventing or ameliorating (hyper)proliferative diseases and/or disorders responsive to induction of apoptosis, such as e.g. benign or malignant neoplasia, e.g. cancer, particularly any of those cancer diseases mentioned herein, in a patient comprising administering a combination according to the present invention.

In addition, the present invention further relates to the use of a composition, combination, formulation, preparation or kit according to this invention in the manufacture of a pharmaceutical product, such as e.g. a commercial package or a medicament, for treating, preventing, or ameliorating (hyper)proliferative diseases, such as e.g. cancer, and/or disorders responsive to the induction of apoptosis, particularly those diseases mentioned herein, such as e.g. malignant or benign neoplasia.

The present invention further relates to a commercial package comprising one or more compounds of the present invention together with instructions for simultaneous, concurrent, sequential or separate use with one or more chemotherapeutic and/or target specific anti-cancer agents, such as e.g. any of those mentioned herein.

The present invention further relates to a commercial package consisting essentially of one or more compounds of the present invention as sole active ingredient together with instructions for simultaneous, concurrent, sequential or separate use with one or more chemotherapeutic and/or target specific anti-cancer agents, such as e.g. any of those mentioned herein.

The present invention further relates to a commercial package comprising one or more chemotherapeutic and/or target specific anti-cancer agents, such as e.g. any of those mentioned herein, together with instructions for simultaneous, concurrent, sequential or separate use with one or more compounds according to the present invention.

The compositions, combinations, preparations, formulations, kits or packages mentioned in the context of the combination therapy according to this invention may also include more than one of the compounds according to this invention and/or more than one of the art-known anti-cancer agents mentioned.

The first and second active ingredient of a combination or kit-of-parts according to this invention may be provided as separate formulations (i.e. independently of one another), which are subsequently brought together for simultaneous, concurrent, sequential, separate or chronologically staggered use in combination therapy; or packaged and presented together as separate components of a combination pack for simultaneous, sequential, concurrent, separate or chronologically staggered use in combination therapy.

The type of pharmaceutical formulation of the first and second active ingredient of a combination or kit-of-parts according to this invention can be similar, i.e. both ingredients are formulated in separate tablets or capsules, or can be different, i.e. suited for different administration forms, such as e.g. one active ingredient is formulated as tablet or capsule and the other is formulated for e.g. intravenous administration.

The amounts of the first and second active ingredients of the combinations, compositions or kits according to this invention may together comprise a therapeutically effective amount for the treatment, prophylaxis or amelioration of a (hyper)proliferative diseases and/or a disorder responsive to the induction of apoptosis, particularly one of those diseases mentioned herein, such as e.g. malignant or benign neoplasia, especially cancer, like any of those cancer diseases mentioned herein.

In addition, compounds according to the present invention can be used in the pre- or post-surgical treatment of cancer.

In further addition, compounds of the present invention can be used in combination with radiation therapy.

A combination according to this invention can refer to a composition comprising both the compound(s) according to this invention and the other active anti-cancer agent(s) in a fixed combination (fixed unit dosage form), or a medicament pack comprising the two or more active ingredients as discrete separate dosage forms (non-fixed combination). In case of a medicament pack comprising the two or more active ingredients, the active ingredients are preferably packed into blister cards which are suited for improving compliance.

Each blister card preferably contains the medicaments to be taken on one day of treatment. If the medicaments are to be taken at different times of day, the medicaments can be disposed in different sections on the blister card according to the different ranges of times of day at which the medicaments are to be taken (for example morning and evening or morning, midday and evening). The blister cavities for the medicaments to be taken together at a particular time of day are accommodated in the respective range of times of day. The various times of day are, of course, also put on the blister in a clearly visible way. It is also possible, of course, for example to indicate a period in which the medicaments are to be taken, for example stating the times.

The daily sections may represent one line of the blister card, and the times of day are then identified in chronological sequence in this column.

Medicaments which must be taken together at a particular time of day are placed together at the appropriate time on the blister card, preferably a narrow distance apart, allowing them to be pushed out of the blister easily, and having the effect that removal of the dosage form from the blister is not forgotten.

Biological Investigations

The anti-proliferative/cytotoxic activity of the compounds described herein, can be tested on NCl-H460 non-small cell lung cancer cells using the Alamar Blue cell viability assay (described in O'Brien et al. Eur J Biochem 267, 5421-5426, 2000). The compounds are dissolved as 20 mM solutions in dimethylsulfoxide (DMSO) and subsequently diluted in semi-logarithmic steps. DMSO dilutions are further diluted 1:10 into Dulbecco's modified Eagle's medium (DMEM) containing 10% fetal calf serum to a final concentration ten times as much as the final concentration in the test. NCl-H460 cells are seeded into 96 well flat bottom plates at a density of 4000 cells per well in a volume of 180 μl per well. 24 hours after seeding the 20 μl each of the compound dilutions in DMEM medium are added into each well of the 96 Well plate. Each compound dilution is tested as quadruplicates. Wells containing untreated control cells are filled with 50 μl DMEM medium containing 1% DMSO. The cells are then incubated with the substances for 72 hours at 37° C. in a humified atmosphere containing 5% carbon dioxide. To determine the viability of the cells, 20 μl of an Alamar Blue solution (Biosource) are added and the fluorescence is measured at an extinction of 544 nm and an emission of 590 nm. For the calculation of cell viability the emission value from untreated cells is set to 100% viability and the emission rates of treated cells are set in relation to the values of untreated cells. Viabilities are expressed as % values.

The corresponding IC₅₀ values of the compounds for anti-proliferative/cytotoxic activity are determined from the concentration-effect curves.

Representative IC₅₀ values for anti-proliferation/cytotoxicity determined in the aforementioned assay follow from the following table A, in which the numbers of the compound correspond to the numbers of the examples.

TABLE A
Anti-proliferative/cytotoxic activity

| | −log IC₅₀ NCI-H460 |
|---|---|
| Compounds | (mol/l) |
| 1, and 3 to 6 | The inhibitory |
| | values of these |
| | listed Examples lie |
| | in the range from |
| | 5.8 to 6.8 |

The induction of apoptosis can be measured by using a Cell death detection ELISA (Roche Biochemicals, Mannheim, Germany). NCl-H460 cells are seeded into 96 well flat bottom plates at a density of 10000 cells per well in a volume of 50 μl per well. 24 hours after seeding the 50 μl each of the compound dilutions in DMEM medium are added into each well of the 96 Well plate. Each compound dilution is tested at least as triplicates. Wells containing untreated control cells are filled with 50 μl DMEM medium containing 1% DMSO. The cells are then incubated with the substances for 24 hours at 37° C. in a humidified athmosphere containing 5% carbon dioxide. As a positive control for the induction of apoptosis, cells are treated with 50 μM Cisplatin (Gry Pharmaceuticals, Kirchzarten, Germany). Medium is then removed and the cells are lysed in 200 μl lysis buffer. After centrifugation as described by the manufacturer, 10 μl of cell lysate is processed as described in the protocol. The degree of apoptosis is calculated as follows: The absorbance at 405 nm obtained with lysates from cells treated with 50 μM cisplatin is set as 100 cpu (cisplatin units), while an absorbance at 405 nm of 0.0 was set as 0.0 cpu. The degree of apoptosis is expressed as cpu in relation to the value of 100 cpu reached with the lysates obtained from cells treated with 50 μM cisplatin.

In order to assess favorable compound combinations in vitro which might be used for therapeutic applications, a modified Alamar Blue cell viability assay (described in O'Brien et al. Eur J Biochem 267, 5421-5426, 2000) is applied. For that purpose, anti-proliferative/cytotoxic effects of the compounds described herein in combination with other pharmacologically active compounds are assayed on NCl-H460 non-small cell lung cancer cells. The respective compounds are dissolved as 20 mM solutions in dimethylsulfoxide (DMSO) and subsequenty diluted in semi-logarithmic steps. DMSO dilutions are further diluted 1:20 into Dulbecco's modified Eagle's medium (DMEM) containing 10% fetal calf serum to a final concentration ten times as much as the final concentration in the test. NCl-H460 cells are seeded into 96 well flat bottom plates at a density of 4000 cells per well in a volume of 160 μl per well. 24 hours after seeding the 20 μl each of two compound dilutions in DMEM medium are added into each well of the 96 Well plate. Each compound dilution is tested as triplicates. Wells containing untreated control cells are filled with 200 μl DMEM medium containing 1% DMSO. The cells are then incubated with the substances for 72 hours at 37° C. in a humified atmosphere containing 5% carbon dioxide. To determine the viability of the cells, 20 μl of an Alamar Blue solution (Biosource) are added and the fluorescence is measured at an extinction of 544 nm and an emission of 590 nm. For the calculation of cell viability the fluorescence emission value from untreated cells is set to 100% viability and the emission values of treated cells are set in relation to the values of untreated cells. Viabilities are expressed as % values.

In one embodiment, the compounds according to this invention are combined with fixed concentrations of other pharmacologically active compounds. In a special sub-embodiment thereof, these fixed concentrations of other pharmacologically active compounds have no anti-proliferative or cytotoxic effect on their own.

Synergistic or antagonistic effects of compound combinations can be determined from the concentration-effect curves by comparison of curve shapes and IC₅₀ values for single compound treatment vs. the compound combination.

Some compounds which represent analogs of cyclic nucleotides, show synergistic effects with the compounds according to this invention. For example, RP-8-CPT-cAMPs (Biolog Life Science Institute, Bremen, Germany, Reference: Weisskopf et al., Science 265, 1878-1882 (1994)) which is a PDE-resistant inhibitor of protein kinase A (type I and II) is highly synergistic to the compounds described herein. Two other cyclic nucleotide analogs, 8-(4-Chlorophenylthio)-2′-O-methyladenosine-3′,5′-cyclic monophosphate (8-pCPT-2′-O-Me-cAMP) and 8-(4-Methoxyphenylthio)-2′-O-methyladenosine-3′,5′-cyclic monophosphate (8-pMeOPT-2′-O-Me-cAMP) which were described as EPAC (exchange protein activated by cyclic AMP) or CAMP-GEF agonists (Biolog Life Science Institute, Bremen, Germany;References: Enserink, J. M., et al., Nature Cell Biol., 4, 901-906 (2002); Kang, G. et al., J. Biol. Chem., 278, 8279-8285 (2003); Christensen, et al., J. Biol. Chem., 278, 35394-35402 (2003)) display similar synergistic properties.

1. A compound of formula I 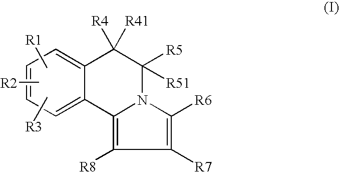
in which
R1 is halogen, nitro, amino, mono- or di-1-4C-alkylamino, 1-4C-alkyl, hydroxyl, 1-4C-alkoxy, 1-4C-alkoxy-2-4C-alkoxy, 3-7C-cycloalkoxy, 3-7C-cycloalkylmethoxy, or completely or predominantly fluorine-substituted 1-4C-alkoxy,
R2 is hydrogen, halogen or 1-4C-alkoxy,
R3 is hydrogen or 1-4C-alkoxy, or
R2 and R3 bound to the benzo ring moiety in ortho-position to each other together form a 1-2C-alkylenedioxy bridge, or
R2 and R3 bound to the benzo ring moiety in ortho-position to each other together form a completely or predominantly fluorine-substituted 1-2C-alkylenedioxy bridge, or
R1 and R2 bound to the benzo ring moiety in ortho-position to each other together form a 1-2C-alkylenedioxy bridge and R3 is hydrogen, or
R1 and R2 bound to the benzo ring moiety in ortho-position to each other together form a completely or predominantly fluorine-substituted 1-2C-alkylenedioxy bridge and R3 is hydrogen,
R4 is hydrogen, or 1-4C-alkyl,
R41 is hydrogen, or 1-4C-alkyl,
R5 is hydrogen,
R51 is hydrogen,
R6 is 1-6C-alkyl, or 1-4C-alkyl substituted by R61, in which
R61 is 1-4C-alkoxycarbonyl, or carboxyl,
R7 is phenyl, naphthyl, Har, R71- and/or R72- and/or R73-substituted phenyl, or R74-substituted Har, in which
Har is bonded to the pyrroloisoquinoline scaffold via a ring carbon atom, and is a monocyclic or fused bicyclic 5- to 10-membered partially or fully aromatic heterocyclic ring radical comprising one to four heteroatoms, each of which is selected from the group consisting of nitrogen, oxygen and sulfur,
R71 is hydroxyl, halogen, nitro, cyano, trifluoromethyl, 1-4C-alkyl, 1-4C-alkoxy, 3-7C-cycloalkoxy, 3-7C-cycloalkylmethoxy, amino, mono- or di-1-4C-alkylamino, 1-4C-alkylsulphonylamino, arylsulphonylamino, mono- or di-1-4C-alkylaminocarbonyl, completely or predominantly fluorine-substituted 1-4C-alkoxy, carbamoyl, tetrazolyl, 1-4C-alkoxycarbonyl, carboxyl, aryl, aryloxy, or —N(H)S(O)₂—N(R712)R713, in which
aryl is phenyl or R711-substituted phenyl, in which
R711 is halogen, 1-4C-alkyl, 1-4C-alkoxy, nitro or cyano,
R712 is 1-4C-alkyl,
R713 is 1-4C-alkyl, or
R712 and R713 together and with inclusion of the nitrogen atom to which they are bound form a radical Het, in which
Het is pyrrolidin-1-yl, piperidin-1-yl or morpholin-4-yl,
R72 is halogen, 1-4C-alkyl, or 1-4C-alkoxy,
R73 is 1-4C-alkyl, or 1-4C-alkoxy,
R74 is halogen, 1-4C-alkyl, trifluoromethyl, 1-4C-alkoxy, cyano, amino, mono- or di-1-4C-alkylamino, 1-4C-alkoxycarbonyl, morpholino, carboxyl, nitro, phenyl, phenoxy, phenyl-1-4C-alkyl, arylsulphonyl, 1-4C-alkylsulphonyl, or —S(O)₂—N(R712)R713,
R8 is —C(O)—R9, in which
R9 is 1-4C-alkyl, 3-7C-cycloalkyl, 3-7C-cycloalkyl-1-4C-alkyl, or phenyl-1-4C-alkyl,
or a stereoisomer, salt or stereoisomer of a salt thereof.

2. A compound of formula I according to claim 1, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is hydrogen, halogen or 1-4C-alkoxy,
R3 is hydrogen or 1-4C-alkoxy,
R4 is hydrogen, or 1-4C-alkyl,
R41 is hydrogen, or 1-4C-alkyl,
R5 is hydrogen,
R51 is hydrogen,
R6 is 1-6C-alkyl, or 1-4C-alkyl substituted by R61, in which
R61 is 1-4C-alkoxycarbonyl, or carboxyl,
R7 is phenyl, naphthyl, Har, R71- and/or R72- and/or R73-substituted phenyl, or R74-substituted Har, in which
Har is bonded to the pyrroloisoquinoline scaffold via a ring carbon atom, and is a monocyclic or fused bicyclic 5- to 10-membered partially or fully aromatic heterocyclic ring radical comprising one to four heteroatoms, each of which is selected from the group consisting of nitrogen, oxygen and sulfur,
R71 is hydroxyl, halogen, nitro, 1-4C-alkoxy, amino, mono- or di-1-4C-alkylamino, carbamoyl, or aryl, in which
aryl is phenyl, or R711-substituted phenyl, in which
R711 is halogen, 1-4C-alkyl, 1-4C-alkoxy, nitro or cyano,
R72 is 1-4C-alkyl, or 1-4C-alkoxy,
R73 is 1-4C-alkyl, or 1-4C-alkoxy,
R74 is 1-4C-alkyl,
R8 is —C(O)—R9, in which
R9 is 1-4C-alkyl, 3-7C-cycloalkyl, 3-7C-cycloalkyl-1-4C-alkyl, or phenyl-1-4C-alkyl,
or a stereoisomer, salt or stereoisomer of a salt thereof.

3. A compound of formula I according to claim 1, in which
either
R1 is nitro, amino, 1-4C-alkyl, 1-4C-alkoxy, 1-4C-alkoxy-2-4C-alkoxy, or completely or predominantly fluorine-substituted 1-4C-alkoxy, and
R2 is 1-4C-alkoxy,
or
R1 is 1-4C-alkoxy, or 1-4C-alkoxy-2-4C-alkoxy, and
R2 is halogen,
R3 is hydrogen,
whereby none of R1, R2 and R3 is bound to the 10-position of the pyrrolo[2,1-a]isoquinoline ring,
R4 is hydrogen, or 1-4C-alkyl,
R41 is hydrogen, or 1-4C-alkyl,
R5 is hydrogen,
R51 is hydrogen,
R6 is 1-6C-alkyl, or 1-4C-alkyl substituted by R61, in which
R61 is 1-4C-alkoxycarbonyl, or carboxyl,
R7 is naphthyl, Har, R71- and/or R72- and/or R73-substituted phenyl, or R74-substituted Har, in which
Har is either
a monocyclic 5-membered heteroaryl radical comprising one to four heteroatoms, each of which is selected from the group consisting of nitrogen, oxygen and sulfur,
or
a monocyclic 6-membered heteroaryl radical comprising one or two nitrogen atoms,
or
a fused bicyclic 9- or 10-membered heteroaryl comprising one to three heteroatoms, each of which is selected from the group consisting of nitrogen, oxygen and sulfur,
or
N-oxy-pyridyl,
R71 is hydroxyl, halogen, nitro, 1-4C-alkoxy, amino, mono- or di-1-4C-alkylamino, carbamoyl, or aryl, in which
aryl is phenyl, or R711-substituted phenyl, in which
R711 is halogen, 1-4C-alkyl, 1-4C-alkoxy, nitro or cyano,
R72 is 1-4C-alkyl, or 1-4C-alkoxy,
R73 is 1-4C-alkyl, or 1-4C-alkoxy,
R74 is 1-4C-alkyl,
R8 is —C(O)—R9, in which
R9 is 1-4C-alkyl, or 3-7C-cycloalkyl,
or a stereoisomer, salt or stereoisomer of a salt thereof.

4. A compound of formula I according to claim 1, in which
either
R1 is 1-4C-alkoxy, 1-4C-alkoxy-2-4C-alkoxy, or completely or predominantly fluorine-substituted 1-4C-alkoxy, and
R2 is 1-4C-alkoxy,
or
R1 is 1-4C-alkoxy, or 1-4C-alkoxy-2-4C-alkoxy, and
R2 is fluorine or chlorine,
R3 is hydrogen,
whereby none of R1, R2 and R3 is bound to the 10-position of the pyrrolo[2,1-a]isoquinoline ring,
R4 is hydrogen, or 1-4C-alkyl,
R41 is hydrogen, or 1-4C-alkyl,
R5 is hydrogen,
R51 is hydrogen,
R6 is 1-4C-alkyl, or 1-4C-alkyl substituted by R61, in which
R61 is 1-4C-alkoxycarbonyl, or carboxyl,
R7 is naphthyl, Har, or R71- and/or R72- and/or R73-substituted phenyl, in which
Har is a fused bicyclic 9- or 10-membered heteroaryl radical comprising one to three heteroatoms, each of which is selected from the group consisting of nitrogen, oxygen and sulfur,
R71 is hydroxyl, halogen, di-1-4C-alkylamino, or aryl, in which
aryl is phenyl, or R711-substituted phenyl, in which
R711 is halogen, 1-4C-alkyl, 1-4C-alkoxy, nitro or cyano,
R72 is 1-4C-alkyl, or 1-4C-alkoxy,
R73 is 1-4C-alkyl, or 1-4C-alkoxy,
R8 is —C(O)—R9, in which
R9 is 1-4C-alkyl, or 3-5C-cycloalkyl,
or a stereoisomer, salt or stereoisomer of a salt thereof.

5. A compound of formula I according to claim 1, in which
either
R1 is 1-2C-alkoxy, 1-2C-alkoxy-2-3C-alkoxy, or completely or predominantly fluorine-substituted 1-2C-alkoxy, and
R2 is 1-2C-alkoxy,
or
R1 is 1-2C-alkoxy, and
R2 is fluorine or chlorine,
R3 is hydrogen,
whereby none of R1, R2 and R3 is bound to the 10-position of the pyrrolo[2,1-a]isoquinoline ring,
R4 is hydrogen, or methyl,
R41 is hydrogen, or methyl,
R5 is hydrogen,
R51 is hydrogen,
R6 is methyl,
R7 is either
naphthyl,
or
dimethyamino-phenyl,
or
4-hydroxy-3,5-dimethylphenyl,
or
2-fluoro-3,4-dimethoxy-phenyl,
or
3,4,5-trimethoxy-phenyl,
or
1,1′-biphen-4-yl,
or
Har, in which
Har is a fused bicyclic 9- or 10-membered heteroaryl comprising a benzene ring and one or two heteroatoms, each of which is selected from the group consisting of nitrogen, oxygen and sulfur,
R8 is —C(O)—R9, in which
R9 is 1-2C-alkyl, or cyclopropyl,
or a stereoisomer, salt or stereoisomer of a salt thereof.

6. A compound according to claim 1, which has formula Ia or formula Ib, 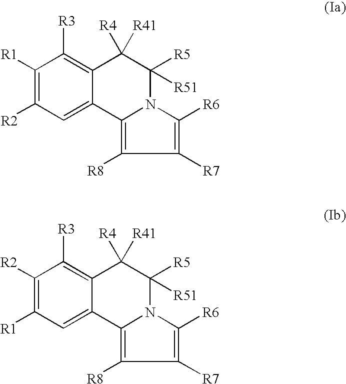
in which
R5 and R51 are both hydrogen,
R6 is methyl, and
R1, R2, R3, R4, R41 and R8 have any of the meanings indicated below:

| R1 | R2 | R3 | R4 | R41 | R8 |
|---|---|---|---|---|---|
| —OCH₃ | —OCH₃ | H | H | H | ethylcarbonyl |
| —OCF₂H | —OCH₃ | H | H | H | ethylcarbonyl |
| —Cl | —OCH₃ | H | H | H | ethylcarbonyl |
| —F | —OCH₃ | H | H | H | ethylcarbonyl |
| —O(CH₂)₂OCH₃ | —OCH₃ | H | H | H | ethylcarbonyl |
| —OCH₂CH₃ | —OCH₃ | H | H | H | ethylcarbonyl |
| —OCH₃ | —OCH₃ | H | H | H | cyclopropyl- |
| | | | | | carbonyl |
| —OCF₂H | —OCH₃ | H | H | H | cyclopropyl- |
| | | | | | carbonyl |
| —Cl | —OCH₃ | H | H | H | cyclopropyl- |
| | | | | | carbonyl |
| —F | —OCH₃ | H | H | H | cyclopropyl- |
| | | | | | carbonyl |
| —O(CH₂)₂OCH₃ | —OCH₃ | H | H | H | cyclopropyl- |
| | | | | | carbonyl |
| —OCH₂CH₃ | —OCH₃ | H | H | H | cyclopropyl- |
| | | | | | carbonyl |
| —OCH₃ | —OCH₃ | H | —CH₃ | H | ethylcarbonyl |
| —OCF₂H | —OCH₃ | H | —CH₃ | H | ethylcarbonyl |
| —Cl | —OCH₃ | H | —CH₃ | H | ethylcarbonyl |
| —F | —OCH₃ | H | —CH₃ | H | ethylcarbonyl |
| —O(CH₂)₂OCH₃ | —OCH₃ | H | —CH₃ | H | ethylcarbonyl |
| —OCH₂CH₃ | —OCH₃ | H | —CH₃ | H | ethylcarbonyl |
| —OCH₃ | —OCH₃ | H | —CH₃ | H | cyclopropyl |
| | | | | | carbonyl |
| —OCF₂H | —OCH₃ | H | —CH₃ | H | cyclopropyl- |
| | | | | | carbonyl |
| —Cl | —OCH₃ | H | —CH₃ | H | cyclopropyl- |
| | | | | | carbonyl |
| —F | —OCH₃ | H | —CH₃ | H | cyclopropyl- |
| | | | | | carbonyl |
| —O(CH₂)₂OCH₃ | —OCH₃ | H | —CH₃ | H | cyclopropyl- |
| | | | | | carbonyl |
| —OCH₂CH₃ | —OCH₃ | H | —CH₃ | H | cyclopropyl- |
| | | | | | carbonyl |
| —OCH₃ | —OCH₃ | H | —CH₃ | —CH₃ | ethylcarbonyl |
| —OCF₂H | —OCH₃ | H | —CH₃ | —CH₃ | ethylcarbonyl |
| —Cl | —OCH₃ | H | —CH₃ | —CH₃ | ethylcarbonyl |
| —F | —OCH₃ | H | —CH₃ | —CH₃ | ethylcarbonyl |
| —O(CH₂)₂OCH₃ | —OCH₃ | H | —CH₃ | —CH₃ | ethylcarbonyl |
| —OCH₂CH₃ | —OCH₃ | H | —CH₃ | —CH₃ | ethylcarbonyl |
| —OCH₃ | —OCH₃ | H | —CH₃ | —CH₃ | cyclopropyl- |
| | | | | | carbonyl |
| —OCF₂H | —OCH₃ | H | —CH₃ | —CH₃ | cyclopropyl- |
| | | | | | carbonyl |
| —Cl | —OCH₃ | H | —CH₃ | —CH₃ | cyclopropyl- |
| | | | | | carbonyl |
| —F | —OCH₃ | H | —CH₃ | —CH₃ | cyclopropyl- |
| | | | | | carbonyl |
| —O(CH₂)₂OCH₃ | —OCH₃ | H | —CH₃ | —CH₃ | cyclopropyl- |
| | | | | | carbonyl |
| —OCH₂CH₃ | —OCH₃ | H | —CH₃ | —CH₃ | cyclopropyl- |
| | | | | | carbonyl |
| —OCH₃ | —OCH₂CH₃ | H | H | H | ethylcarbonyl |
| —OCF₂H | —OCH₂CH₃ | H | H | H | ethylcarbonyl |
| —Cl | —OCH₂CH₃ | H | H | H | ethylcarbonyl |
| —F | —OCH₂CH₃ | H | H | H | ethylcarbonyl |
| —O(CH₂)₂OCH₃ | —OCH₂CH₃ | H | H | H | ethylcarbonyl |
| —OCH₂CH₃ | —OCH₂CH₃ | H | H | H | ethylcarbonyl |
| —OCH₃ | —OCH₂CH₃ | H | H | H | cyclopropyl- |
| | | | | | carbonyl |
| —OCF₂H | —OCH₂CH₃ | H | H | H | cyclopropyl- |
| | | | | | carbonyl |
| —Cl | —OCH₂CH₃ | H | H | H | cyclopropyl- |
| | | | | | carbonyl |
| —F | —OCH₂CH₃ | H | H | H | cyclopropyl- |
| | | | | | carbonyl |
| —O(CH₂)₂OCH₃ | —OCH₂CH₃ | H | H | H | cyclopropyl- |
| | | | | | carbonyl |
| —OCH₂CH₃ | —OCH₂CH₃ | H | H | H | cyclopropyl- |
| | | | | | carbonyl |
| —OCH₃ | —OCH₂CH₃ | H | —CH₃ | H | ethylcarbonyl |
| —OCF₂H | —OCH₂CH₃ | H | —CH₃ | H | ethylcarbonyl |
| —Cl | —OCH₂CH₃ | H | —CH₃ | H | ethylcarbonyl |
| —F | —OCH₂CH₃ | H | —CH₃ | H | ethylcarbonyl |
| —O(CH₂)₂OCH₃ | —OCH₂CH₃ | H | —CH₃ | H | ethylcarbonyl |
| —OCH₂CH₃ | —OCH₂CH₃ | H | —CH₃ | H | ethylcarbonyl |
| —OCH₃ | —OCH₂CH₃ | H | —CH₃ | H | cyclopropyl- |
| | | | | | carbonyl |
| —OCF₂H | —OCH₂CH₃ | H | —CH₃ | H | cyclopropyl- |
| | | | | | carbonyl |
| —Cl | —OCH₂CH₃ | H | —CH₃ | H | cyclopropyl- |
| | | | | | carbonyl |
| —F | —OCH₂CH₃ | H | —CH₃ | H | cyclopropyl- |
| | | | | | carbonyl |
| —O(CH₂)₂OCH₃ | —OCH₂CH₃ | H | —CH₃ | H | cyclopropyl- |
| | | | | | carbonyl |
| —OCH₂CH₃ | —OCH₂CH₃ | H | —CH₃ | H | cyclopropyl- |
| | | | | | carbonyl |
| —OCH₃ | —OCH₂CH₃ | H | —CH₃ | —CH₃ | ethylcarbonyl |
| —OCF₂H | —OCH₂CH₃ | H | —CH₃ | —CH₃ | ethylcarbonyl |
| —Cl | —OCH₂CH₃ | H | —CH₃ | —CH₃ | ethylcarbonyl |
| —F | —OCH₂CH₃ | H | —CH₃ | —CH₃ | ethylcarbonyl |
| —O(CH₂)₂OCH₃ | —OCH₂CH₃ | H | —CH₃ | —CH₃ | ethylcarbonyl |
| —OCH₂CH₃ | —OCH₂CH₃ | H | —CH₃ | —CH₃ | ethylcarbonyl |
| —OCH₃ | —OCH₂CH₃ | H | —CH₃ | —CH₃ | cyclopropyl- |
| | | | | | carbonyl |
| —OCF₂H | —OCH₂CH₃ | H | —CH₃ | —CH₃ | cyclopropyl- |
| | | | | | carbonyl |
| —Cl | —OCH₂CH₃ | H | —CH₃ | —CH₃ | cyclopropyl- |
| | | | | | carbonyl |
| —F | —OCH₂CH₃ | H | —CH₃ | —CH₃ | cyclopropyl- |
| | | | | | carbonyl |
| —O(CH₂)₂OCH₃ | —OCH₂CH₃ | H | —CH₃ | —CH₃ | cyclopropyl- |
| | | | | | carbonyl |
| —OCH₂CH₃ | —OCH₂CH₃ | H | —CH₃ | —CH₃ | cyclopropyl- |
| | | | | | carbonyl |

or a stereoisomer, salt or stereoisomer of a salt thereof.

7. A compound of formula I according to claim 1, comprising one or more of the following:
either
R1 is bonded in the 8-position of the pyrrolo[2.1-a]isoquinoline ring, and is chlorine, 2-methoxy-ethoxy or difluoromethoxy, and
R2 is bonded in the 9-position of the pyrrolo[2,1-a]isoquinoline ring, and is 1-2C-alkoxy,
or
R1 is bonded in the 9-position of the pyrrolo[2,1-a]isoquinoline ring, and is chlorine, fluorine or difluoromethoxy, and
R2 is bonded in the 8-position of the pyrrolo[2,1-a]isoquinoline ring, and is 1-2C-alkoxy,
and
R3 is hydrogen;
R4 is hydrogen or methyl,
R41 is hydrogen or methyl,
R5 is hydrogen, and
R51 is hydrogen;
R6 is methyl;
R7 is naphthyl, quinolinyl, benzothiophenyl, or indolyl,
or
3-dimethyamino-phenyl, 4-hydroxy-3,5-dimethylphenyl or 1,1′-biphen-4-yl;
and
R8 is ethylcarbonyl or cyclopropylcarbonyl;
or a stereoisomer, salt or stereoisomer of a salt thereof.

8. (canceled)

9. (canceled)

10. A pharmaceutical composition comprising one or more compounds according to claim 1, or a pharmaceutically acceptable stereoisomer, salt or stereoisomer of a salt thereof together with a pharmaceutically acceptable excipient and/or vehicle.

11. A method for treating, preventing or ameliorating a (hyper)proliferative disease of benign or malignant behaviour and/or a disorder responsive to the induction of apoptosis in a patient comprising administering to said patient a therapeutically effective amount of a compound according to claim 1, or a pharmaceutically acceptable stereoisomer, salt or stereoisomer of a salt thereof.

12. A combination comprising
a first active ingredient, which is at least one compound according to claim 1, or a pharmaceutically acceptable stereoisomer, salt or stereoisomer of a salt thereof, and
a second active ingredient, which is at least one anti-cancer agent selected from the group consisting of chemotherapeutic anti-cancer agents, target-specific anti-cancer agents and agents which interfere with cyclic nucleotide metabolism, for separate, sequential, simultaneous, concurrent or chronologically staggered use in therapy.

13. A method for treating, preventing or ameliorating a hyperproliferative disease and/or disorder responsive to induction of apoptosis in a patient comprising administering separately, simultaneously, concurrently, sequentially or chronologically staggered to said patient in need thereof an amount of a first active compound, which is a compound according to claim 1, or a pharmaceutically acceptable stereoisomer, salt or stereoisomer of a salt thereof, and
an amount of at least one second active compound, said second active compound being an anti-cancer agent selected from the group consisting of chemotherapeutic anti-cancer agents, target-specific anti-cancer agents and agents which interfere with cyclic nucleotide metabolism,
wherein the amounts of the first active compound and said second active compound result in a therapeutic effect.

14. The combination according to claim 12, in which said chemotherapeutic anti-cancer agents are selected from the group consisting of (i) alkylating/carbamylating agents; (ii) platinum derivatives; (iii) antimitotic agents/tubulin inhibitors; (iv) topoisomerase inhibitors; (v) pyrimidine antagonists; (vi) purin antagonists; and (vii) folic acid antagonists.

15. The combination according to claim 12, in which said target-specific anti-cancer agents are selected from the group consisting of (i) kinase inhibitors; (ii) proteasome inhibitors; (iii) histone deacetylase inhibitors; (iv) heat shock protein 90 inhibitors; (v) vascular targeting agents (VAT); (vi) monoclonal antibodies; (vii) oligonucleotide based therapeutics; (viii) Toll-like receptor/TLR 9 agonists; (ix) protease inhibitors; and (x) hormonal therapeutics.

16. The combination according to claim 12, in which said agents which interfere with cyclic nucleotide metabolism are selected from the group consisting of phosphodiesterase inhibitors, protein kinase A or protein kinase G agonists or antagonists, activators or inhibitors of exchange protein activated by cyclic AMP (Epac) or cAMP-GEF, and activators or inhibitors of guanylate cyclase or adenylate cyclase.

17. The method or combination according to claim 11, wherein the disease or disorder is selected from the group consisting of cancer of the breast, bladder, bone, brain, central and peripheral nervous system, colon, endocrine glands, esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva; inherited cancers, retinomblastoma and Wilms tumor; leukemia, lymphoma, non-Hodgkins disease, chronic and acute myeloid leukaemia, acute lymphoblastic leukemia, Hodgkins disease, multiple myeloma and T-cell lymphoma; myelodysplastic syndrome, plasma cell neoplasia, paraneoplastic syndromes, cancers of unknown primary site and AIDS related malignancies.
